(19) United States
(12) Patent Application Publication
(10) Pub. No.: US 2025/0213729 A1
(43) Pub. Date: Jul. 3, 2025

(54) METHOD TO STIMULATE REGENERATION OF RETINAL GANGLION CELLS

(71) Applicant: UNIVERSITY OF WASHINGTON, SEATTLE, WA (US)

(72) Inventors: Levi J. Todd, SEATTLE, WA (US); Thomas A. Reh, SEATTLE, WA (US); Wesley Jenkins, SEATTLE, WA (US); Marina Pavlou, SEATTLE, WA (US); Juliette Wohlschlegel, SEATTLE, WA (US)

(73) Assignee: UNIVERSITY OF WASHINGTON, SEATTLE, WA (US)

(21) Appl. No.: 18/852,391

(22) PCT Filed: Mar. 31, 2023
(86) PCT No.: PCT/US23/65219

§ 371 Date: Sep. 27, 2024

Related U.S. Application Data
(60) Provisional application No. 63/362,361, filed on Apr. 1, 2022.

(51) Int. Cl. A61K 48/00; C07K 14/47; C12N 15/86

(52) U.S. Cl. CPC A61K 48/0058; C07K 14/47; C12N 15/86; C12N 2750/14143; C12N 2830/008

(57) ABSTRACT

Compositions, nucleic acid molecules and methods for inducing retinal regeneration and reprogramming of Müller glia (MG) into retinal ganglion cells in a subject are described. Developmental retinal ganglion cell (RGC) transcription factors Pou4f2 and Islet1 increase the Ascl1-induced neurogenic capacity of MG. The combination of Ascl1, Pou4f2 and Islet1 stimulates MG to generate bipolar cells and RGC-like neurons. Likewise, the transcription factor Onecut1, which is expressed in developing retinal cells, but not in MG, induces MG to generate RCG-like cells. Additional transcription factors that can be used include Irx2, Irx5, Neurod2, Ebf1, and Tcf3. MG-derived RGCs can exhibit action potentials in vivo, and display chromatin profiles similar to developing RGCs.

This application claims benefit of U.S. provisional patent application No. 63/362,361, filed Apr. 1, 2022, the entire contents of which are incorporated by reference into this application.

### ACKNOWLEDGEMENT OF GOVERNMENT SUPPORT

This invention was made with government support under Grant No. R01 EY021482, awarded by the National Institutes of Health (NIH). The government has certain rights in the invention.

### REFERENCE TO A SEQUENCE LISTING

The content of the XML file of the sequence listing named “UW77_seq”, which is 666 kb in size, created on Mar. 30, 2023, and electronically submitted herewith the application, is incorporated herein by reference in its entirety.

### BACKGROUND

Neurodegenerative disorders of the eye result in blindness because the mammalian nervous system lacks a regenerative capacity. In other vertebrates, such as fish and amphibians, the retina is able to replace lost neurons and restore visual function. Müller glia (MG), the primary glial cell in the vertebrate retina, can serve as a source of neurogenic progenitors in regenerative species. However, in the mammalian retina, MG respond to retinal damage by undergoing an inflammatory response instead of a regenerative one.

There are numerous diseases that cause the loss of specific neuronal populations in the retina resulting in blindness. As such, there remains a need to stimulate regeneration in the human retina for the development of new types of regenerative therapies for patients.

### SUMMARY

Described herein are compositions, nucleic acid molecules and methods for inducing retinal regeneration and reprogramming of Müller glia (MG) into retinal ganglion cells in a subject. Examples provided herein show that the developmental retinal ganglion cell (RGC) transcription factors Pou4f2 and Islet1 increase the Ascl1-induced neurogenic capacity of MG. The combination of Ascl1, Pou4f2 and Islet1 stimulates MG to generate bipolar cells and RGC-like neurons. Likewise, the transcription factor Onecut1, which is expressed in developing retinal cells, but not in MG, induces MG to generate RCG-like cells. Additional transcription factors that can be used include Irx2, Irx5, Neurod2, Ebf1, and Tcf3. MG-derived RGCs can exhibit action potentials in vivo, and display chromatin profiles similar to developing RGCs.

Disclosed herein is a nucleic acid molecule comprising a nucleic acid sequence encoding a developmental retinal ganglion cell (RGC) transcription factor, wherein the RGC transcription factor is selected from the group consisting of Onecut1, Pou4f2, Islet1, Irx2, Irx5, Neurod2, Ebf1, and Tcf3, and combinations thereof. In some embodiments, the transcription factor is selected from Onecut1, Pou4f2, and Islet1, and combinations thereof. In some embodiments, the transcription factor is selected from Irx2, Irx5, Neurod2, Ebf1, and Tcf3, and combinations thereof. In some embodiments, the RGC transcription factor is Onecut1. In some embodiments, the RGC transcription factor comprises Pou4f2 and/or Islet1. In some embodiments, the RGC transcription factor comprises Irx2 and/or Neurod2.

In some embodiments, the nucleic acid sequence further comprises a nucleic sequence that encodes a proneural basic helix-loop-helix (bHLH) transcription factor. In some embodiments, the proneural bHLH transcription factor and the RGC transcription factor are expressed as a fusion protein. Representative examples of proneural bHLH transcription factors include, but are not limited to, Ascl1, Atonal7 (also known as Math5), Atoh1 (also known as Math1), Neurogenin-2, and Neuronal Differentiation 1 (Neurod1). In one embodiment, the nucleic acid molecule further comprises a nucleic acid sequence encoding Ascl1 and Atoh1. In another embodiment, the nucleic acid molecule further comprises a nucleic acid sequence encoding Ascl1 and Atoh7. In another embodiment, the nucleic acid molecule further comprises a nucleic acid sequence encoding Atoh1 and Atoh7. In another embodiment, the nucleic acid molecule further comprises a nucleic acid sequence encoding Ascl1, Atoh1 and Atoh7. In some embodiments, the nucleic acid molecule comprises a nucleic acid sequence encoding two, three, four, or all five of Ascl1, Atonal7, Atoh1, Neurogenin-2, and Neurod1.

In some embodiments, the nucleic acid comprises Pou4f2 and/or Islet1 and/or Ascl1. In some embodiments, the nucleic acid comprises Pou4f2, Islet1, and Ascl1.

In some embodiments, the nucleic acid sequence further comprises a promoter sequence in operable linkage with the nucleic acid sequence encoding the RGC transcription factor. In some embodiments, the promoter sequence does not naturally occur in operable linkage with the nucleic acid sequence encoding the RGC transcription factor. In some embodiments, the promoter sequence is for a gene specifically expressed in glial cells, such as RLBP1 or GLAST. In some embodiments, the promoter sequence is a HES1, RLBP1 or GLAST promoter. In some embodiments, the promoter sequence will precede an effector gene CRE or tTA that will drive transcription in an inducible manner. In one embodiment, the MG-specific promoter sequence is a Rbpl1 promoter sequence or a portion thereof. In other embodiments, the nucleic acid sequence comprises an IRES or 2A self-cleaving sites situated between the sequences encoding the transcription factors, for example, in a multicistronic or polycistronic configuration. In other embodiments, the nucleic acid sequence will comprise elements that respond to the presence of CRE or tTA, to trigger transcription of genes in an inducible manner. These elements include, but are not limited to, loxP, lox2272 and tetracycline response element (TRE).

Also provided is a composition comprising a nucleic acid molecule described herein. In some embodiments, the nucleic acid molecule is an mRNA. Such compositions can be formulated for delivery, optionally in the form of a vector. In some embodiments, the vector is a non-viral vector or a viral vector. In some embodiments, the viral vector is an adeno-associated viral (AAV) vector or a lentiviral vector. In some embodiments, the composition is formulated for administration to the retina. Examples of such administration include, but are not limited to, intravitreal or subretinal injection.

In some embodiments, the composition further comprises a histone deacetylase (HDAC) inhibitor (HDACi). Representative examples of the HDACi include, but are not limited to, trichostatin A (TSA), Istodax™ also known as (Pro)/romidepsin, Beleodaq™, also known as (Pro)/belinostat, Farydak™, also known as (Pro)/panobinostat, and Zolinza™, also known as (Pro)/vorinostat. Exemplary HDACi peptides include, without limitation, 16cyc-HxA, 16lin-HxA and 16KA (SEQ ID NO: 58-60).

Also disclosed herein is a method for inducing retinal regeneration, or a method for stimulating regeneration of a retinal ganglion cell by administering to a cell, e.g., an MG cell, a nucleic acid molecule comprising a nucleic acid sequence encoding a developmental retinal ganglion cell (RGC) transcription factor (TF). In certain embodiments the cell is in vitro or ex vivo. Exemplary RGC TFs include Onecut1, Pou4f2, Islet1, Irx2, Irx5, Neurod2, Ebf1, and Tcf3, and combinations thereof. In some embodiments, the transcription factor is selected from Onecut1, Pou4f2, and Islet1, and combinations thereof. In some embodiments, the transcription factor is selected from Irx2, Irx5, Neurod2, Ebf1, and Tcf3, and combinations thereof. In some embodiments, the RGC transcription factor is Onecut1. In some embodiments, the RGC transcription factor comprises Pou4f2 and/or Islet1. In some embodiments, the RGC transcription factor comprises Irx2 and/or Neurod2.

Also disclosed herein is a method for inducing retinal regeneration, or a method for stimulating regeneration of retinal ganglion cells, in a subject. These methods comprise administering to a retina of the subject a nucleic acid molecule comprising a nucleic acid sequence encoding a developmental retinal ganglion cell (RGC) transcription factor. Exemplary RGC TFs include Onecut1, Pou4f2, Islet1, Irx2, Irx5, Neurod2, Ebf1, and Tcf3, and combinations thereof. In some embodiments, the transcription factor is selected from Onecut1, Pou4f2, and Islet1, and combinations thereof. In some embodiments, the transcription factor is selected from Irx2, Irx5, Neurod2, Ebf1, and Tcf3, and combinations thereof. In some embodiments, the RGC transcription factor is Onecut1. In some embodiments, the RGC transcription factor comprises Pou4f2 and/or Islet1. In some embodiments, the RGC transcription factor comprises Irx2 and/or Neurod2.

In some embodiments, the nucleic acid molecule further comprises a nucleic sequence that encodes a proneural basic helix-loop-helix (bHLH) transcription factor, as described herein. In some embodiments, the nucleic acid molecule comprises a first nucleic acid molecule encoding a proneural bHLH transcription factor, and a second nucleic acid molecule encoding a RGC transcription factor. In some embodiments, the nucleic acid molecule is administered in the form of a composition. In some embodiments, the administering comprises a first administration of composition comprising a first nucleic acid molecule encoding a proneural bHLH transcription factor, and a second administration at a subsequent time point of a composition comprising a second nucleic acid molecule encoding a RGC transcription factor selected from the group consisting of Onecut1, Pou4f2, Islet1, Irx2, Irx5, Neurod2, Ebf1, and Tcf3, and combinations thereof. In some embodiments, the transcription factor is selected from Onecut1, Pou4f2, and Islet1, and combinations thereof. In some embodiments, the transcription factor is selected from Irx2, Irx5, Neurod2, Ebf1, and Tcf3, and combinations thereof. In some embodiments, the RGC transcription factor is Onecut1. In some embodiments, the RGC transcription factor comprises Pou4f2 and/or Islet1. In some embodiments, the RGC transcription factor comprises Irx2 and/or Neurod2. In some embodiments, the proneural bHLH transcription factor and the RGC transcription factor are administered as a fusion protein. In some embodiments, the first and/or second nucleic acid molecule is an mRNA.

In additional embodiments, the nucleic acid molecules and methods disclosed herein stimulate production of functional RGCs from reprogrammed MG. In another embodiment, the number of the MG-derived functional RGCs is increased. In another embodiment, the number of functional RGCs is increased by 40%. In another embodiment, the subject is treated for retinal disease, damage or degeneration in the retina. In another embodiment, the subject is an adult. In another embodiment, a vector comprises the nucleic acid molecule. In one embodiment, the vector is a non-viral vector or a viral vector, and the viral vector is an adeno-associated viral (AAV) vector or a lentiviral vector. In an additional embodiment, a promoter sequence is in operable linkage with the nucleic acid encoding the developmental RGC transcription factor. In one embodiment, the promoter is a Cre-inducible or tTA-inducible or MG-specific promoter. In one embodiment, administering to the retina is intravitreal or subretinal injection.

Also provided herein are methods for inducing retinal regeneration comprising administering to a subject a composition as described herein. In some embodiments, the methods are effective to increase the number of Müller glial-derived RGCs, to induce Müller glial cells to enter the mitotic cell cycle, and/or to generate new RGCs. In some embodiments of the method, the number of RGCs increases by at least 40% relative to a baseline level or other reference amount representative of an untreated retina. In some embodiments, the number of RGCs increases by 10%, 20%, 25%, 50%, 100%, 150%, 200%, or more.

The subject in the methods disclosed herein is typically a mammal, such as a human or veterinary subject. In one embodiment, the subject is an adult. The subject, in some embodiments, has a retinal degenerative disease. Examples of such retinal degenerative diseases include, but are not limited to, Age-related macular degeneration, glaucoma, ischemia, central retinal arterial occlusion and inherited retinal diseases, such as Retinitis Pigmentosa or Usher's syndrome.

Reprogramming of MG and regeneration of retinal neurons is particularly important for developing therapeutic products and methods for a range of degenerative ocular diseases such as, for example, and without limitation, retinal degeneration caused by diabetic retinopathy, glaucoma, and age-related macular degeneration. One such retinal degenerative disease is known as central retinal artery occlusion (CRAO), wherein blood flow through the central retinal artery is blocked or occluded often resulting in loss of vision. CRAO is caused by thromboembolus, carotid artery atherosclerosis, giant cell arteritis, aneurysms or arterial spasms. Current treatment paradigms for many of these types of degenerative diseases of the retina, particularly for CRAO, show little to no definitive improvement in outcomes. Another indication might be paracentral acute middle maculopathy, from vein occlusion (CRVO) or other causes that leads to inner retinal thinning and vision loss.

### BRIEF DESCRIPTION OF THE DRAWINGS

FIGS. 1A-1H Pou4f2 and/or Islet1 stimulate regeneration of RGC-like neurons. (1A) Schematic depicting the transgenic constructs used to induce Ascl1 and Pou4f2/Islet1 specifically in MG. Pou4f2/Islet1 is surrounded by mutually exclusive floxed sites, leading to expression of Pou4f2, Islet1, or both in the presence of active Cre. (1B) Experimental paradigm to induce retinal regeneration in adult mice. Tamoxifen (TMX). (1C) Representative sections of the retina after intravitreal NMDA damage, showing transgenic expression of Pou4f2/Bm3 and/or Islet1 in GFP+ lineage-traced MG. DAPI, 4′,6-diamidino-2-phenylindole. (1D) Quantification of the percent of transgene-expressing MG that express Pou4f2, Islet1, or both. (1E and 1F) Representative sections showing MG-derived neurons after the regeneration paradigm expressing HuC/D. (1G) Quantification of the percent of GFP+ MG-derived neurons that express either HuC/D or Otx2. (1H) Examples of MG-derived neurons expressing Otx2. Significance of difference was determined using an unpaired t test (asterisk=p<0.0001); dots represent individual animals. Scale bars, 50 μm. ONL, outer nuclear layer; INL, inner nuclear layer; GCL, ganglion cell layer. Mouse schematic was made with Biorender.com.

FIGS. 2A-2F. Islet1+Pou4f2+Ascl1 (IPA)-stimulated MG-derived neurons display complex neuronal morphology. (A) Retinal whole mounts stained for GFP (MG-derived cells) and Brn3. (B to E) Examples of the morphology of GFP+MG-derived cells. (F) MG-derived (GFP+) cell with complex neurites colabeled with Bm3. Scale bars, 50 μm.

FIGS. 3A-3G. scRNA-seq analysis of Pou4f2/Islet1-stimulated neurons reveals molecular characteristics of RGCs. (A) UMAP plot for FACS-sorted MG-derived cells after the IPA regeneration paradigm combined with a previous scRNA-seq dataset where Ascl1 only was used. (B) Feature plots highlight the major clusters of MG (Rlbp1), neurogenic transition (Bhlhe22), bipolars (Cabp5), photoreceptors (Rcvm), and RGC-like cells (Elavl4 and Sox11). (C) The distribution of cells from each treatment projected onto a split UMAP plot. Donut plots represent the percent each cluster comprises of the dataset. (D) Heatmap comparing scRNA-seq datasets of Ascl1 only versus IPA treatment. Selected genes are depicted that are associated with RGCs. (E) Retinal sections stained for MG-derived cells (GFP) with Satb1 (red), and quantifications show the percent of GFP+ cells that are Satb1+. (F) Retinal sections stained for MG-derived cells (GFP) with Calretinin (red), and quantifications show the percent of GFP+ cells that are Calretinin+. (G) UMAP of IPA-derived neurons integrated with scRNA-seq of E14 mouse retina from Clark et al. (21), revealing that IPA neurons cluster similarly to immature RGCs. Scale bars, 50 μm.

FIGS. 4A-4M. Islet1 and Pou4f2 coinduction stimulates RGC-like neurons from MG in vitro. (A) Schematic of transgenic construct to induce IPA in all primary MG in vitro by doxycycline. (B) Paradigm for inducing Ascl1-mediated neurogenesis in vitro. (C to E) Representative images of EdU+MG-derived neurons expressing neuronal markers.) (C) EdU+ (white) MG-derived cell expressing Tuj1 (red). (D) MG-derived neuron expressing EdU (white), Neurofilament M (NFM; red), and the GFP transgene reporter (GFP). ((E) MG-derived neuron expressing Calbindin (red) colabeling with EdU (white), DAPI, and GFP. (F to H) UMAP plots of cultured MG reprogrammed with Ascl1 (F) and IPA (G), integrated with cells from in vivo IPA regeneration model (H) as reference. (I) Stacked bar plot showing composition of neuronal clusters in each sample. BC, bipolar cell. (J) Feature plots highlighting differentially expressed genes in neuronal clusters of either reprogramming strategy. (K) Highlighted cells of the Ascl1 and IPA datasets used for downstream DGE analysis in (L). (M) Heatmap of genes differentially expressed in either the Ascl1 or IPA condition. WT, untreated cultured MG included for baseline values. Statistics for differential gene analysis: Wilcoxon Mann-Whitney test for significance (P<0.05). Scale bars, 50 μm.

FIGS. 5A-5D. Physiological profiling of IPA-induced neurons. (A) Summary of electrical properties of cells in this study compared to endogenous neurons, endogenous glia, and MG-derived neurons from previous regeneration protocols (4, 5, 13). Resting potential and input resistance were estimated from current clamp recordings. (B) Examples of responses to current (left) and voltage (right) steps for three cells. (C) Three examples of cells that responded to light stimulus. (D) Examples of cells that displayed action potentials or similar events. The two left panels are responses to hyperpolarizing and depolarizing current steps from a cell that generated apparent Na+ spikes. The right two panels are responses from a cell that generated smaller discrete events, likely Ca2+ spikes.

FIGS. 6A-6J. scATAC reveals MG remodel chromatin to an RGC-like state in response to IPA treatment. (A) Combined UMAP of GFP+ sorted MG and their progeny from the in vivo regeneration paradigm with Ascl1-only (B) or IPA treatment (C). (D) Coverage plots for known marker genes used to identify clusters. (E and F) chromVAR scores of Otx2 and Pou4f2 to highlight differential accessibility of their respective motifs. (G) Scatterplot comparing accessible motifs in E14 RGCs versus IPA-induced RGCs. (H) Top “GO biological process” results for peaks specific to E14 RGCs compared to IPA-derived RGC-like neurons. (1) Heatmap of the chromVAR activity scores of the top variable motifs for TFs found on the pseudotime lineage of E14 progenitor cells to RGCs. (J) Heatmap of the same chromVAR activity scores of the E14 motifs plotted on pseudotime from MG to RGC-like neuron after IPA treatment. (K) Retinal sections showing GFP+MG-derived cells costained with the MG nuclei marker Sox2 (red) and quantification of GFP+ cells expressing Sox2. Scale bars, 50 μm.

FIGS. 7A-7I. The addition of Atoh1 to the IPA paradigm facilitates transition from a progenitor state to a differentiated neuron. (A) Schematic of transgenic construct to express IPA with Atoh1 in MG. (B) Regeneration paradigm for inducing IPA:Atoh1 expression in MG in the damaged retina. (C) Representative immunofluorescence images of regenerated neurons from IPA:Atoh1 mice demonstrating MG-derived neurons (GFP+) are HuC/D+ and not Otx2+. (D) Integrated UMAP of FACS-sorted MG-derived cells after regeneration paradigm with either IPA:Atoh1 or IPA-only overexpression. Highlighted are the RGC-like cells from each dataset that were subsetted for further comparative analysis. (E) Scatterplot highlighting differentially expressed genes between the RGC-like cells of the IPA:Atoh1 and IPA-only regeneration paradigms. (F) GO analysis revealed that neurodevelopmental terms containing many retinal progenitor genes were down-regulated in the IPA:Atoh1 dataset versus IPA only. (G) Integrated UMAP of IPA:Atoh1 data as described above with previously generated Ascl1:Atoh1 dataset (13). Highlighting denotes RGC-like cells from each dataset compared in further analysis. (H) Scatterplot highlighting differentially expressed genes between the RGC-like cells of the IPA:Atoh1 and Ascl1:Atoh1 regeneration paradigms. (I) Bar plot of GO terms relating to neurite outgrowth enriched in the IPA:Atoh1 data. Known neuron projection genes listed are up-regulated with IPA:Atoh1 versus Ascl1:Atoh1.

FIGS. 8A-8C. A subset of IPA-derived neurons are derived from proliferating MG. (8A) Experimental paradigm to label diving cells during the regeneration experiment described in FIG. 1. (8B) Representative sections showing MG-derived cells (GFP+) that previously underwent cell division (EdU+). (8C) Representative image showing some MG-derived neurons (GFP+/HuC/D+, upper panels) are the result of proliferating MG (EdU+ lower left panel). Scale bars are 50 μm. Abbreviations: ONL, outer nuclear layer, INL, inner nuclear layer, GCL, ganglion cell layer.

FIGS. 9A-9E. IPA-treatment is most effective at reprogramming MG if induced prior to injury. (9A) Experimental paradigm using the transgenic mouse described in FIG. 1 to test whether Islet1/Pou4f2/Ascl1 is able to reprogram MG in the absence of retinal injury. (9B) Representative pictures of GFP+MG showing no induction of the ganglion/amacrine marker HuC/D (upper right panel) or the bipolar marker Otx2 (lower right panel). (9C) Experimental paradigm to test whether induction of the IPA-factors after NMDA damage and TSA application can induce MG neurogenesis. (9D) Representative pictures of GFP+MG showing some co-labeling of the neuronal markers HuC/D (upper right) and Otx2 (lower right). (9E) Quantification of the percent of MG-derived cells that express HuC/D or Otx2 after IPA-induction prior to injury, after injury, or without injury. Scale bars are 50 μm.

FIGS. 10A-10E. scRNA-seq analysis showing Pou4f2 biases MG-production towards RGC-like neurons. (10A) UMAPs of integrated IPA and Ascl1-only reprogrammed MG highlighting cells expressing either Isl1, Pou4f2, or the combination of both. (10B) Stacked bar graph quantifying the percent of Islet1-only, Pou4f2-only, or Islet1/Pou4f2 double-positive cells that end up as MG-derived bipolars or RGC-like neurons. (10C) Feature plots highlighting Ascl1-expressing cells which get downregulated as they differentiate into bipolar neurons (Otx2+) or RGC-like neurons (Elavl4+). (10D) Heatmap of the top 40 differentially expressed genes between MG and MG-derived neurons found in the GO-terms “axon guidance”, “axonogenesis”, and “axon outgrowth”. (10E) Feature plots of prediction scores from Seurat's label transfer using a reference dataset of randomly sampled cells of each major retinal neuron class and MG, subsetted from mouse retinal development scRNA-seq data²³.

FIGS. 11A-11D. MG-derived RGCs are a stable population overtime. (11A) Combined UMAP of IPA-treated MG from a three and six week end point. (11B) Split UMAP showing the distribution of cells in the UMAP in (a) from each time point. (11C) Stacked bar graph showing the percentages of each cluster of MG and MG-derived neurons from the three week and six week time point. (11D) Analysis of cell death genes found in MG-derived RGC-like neurons. These data show that cell death genes are not significantly increased in MG-derived RCSs from this 3 to 6 week survival period.

FIGS. 12A-12E. MG from IPA mice express reprogramming factors. (12A) Immunofluorescence of IPA MG treated with doxycycline for 5 days showing Ascl1-IRES-GFP, Brn3, and Isl1 co-labeling in most cells. (12B) UMAP of untreated cultured MG with glial and neuronal cell populations labeled. (12C) Markers of MG and various retinal neurons reveal a small population of bipolar and amacrine cells survived dissociation. (12D) UMAP of integrated datasets from IPA overexpression in vivo and in vitro, with Ascl1 overexpression in vitro. (12E) Expression of melanopsin (Opn4) in three datasets featured in (12D). Scale bars are 50 μm. Abbreviations: MG, Müller glia; AC, amacrine cells; BC, bipolar cells; PR, photoreceptors; RGC, retinal ganglion cells.

FIGS. 13A-13F. scATAC-seq of the E14 embryonic mouse retina. (13A) UMAP plot of scATAC-seq from E14 embryonic mouse retina. Chromvar scores show the motif accessibility used to identify the clusters of progenitors (13B, Sox2), retinal ganglion cells (13C, Pou4f2), cones (13D, Otx2), and neurogenic precursors (13E, Ascl1). (13F) Pseudotime subset of the transition of retinal progenitor cells to retinal ganglion cells that is further analyzed in FIG. 6.

FIGS. 14A-14H. The addition of Atoh1 to IPA significantly induces MG-derived RGC-like cells and does not require retinal damage. (14A) Transgenic mouse construct used for induction of Ascl1, Atoh1, Pou4f2, and Islet1. (14B) Paradigm to assay whether induction of all four transcription factors can induce MG-neurogenesis in the absence of retinal injury. (14C) Representative section of a retina after IPAA treatment showing MG-derived (GFP+) cells express the neuronal marker HuC/D. (14D) Quantification of the percent of MG-derived cells that express the RGC-like marker HuC/D or the bipolar marker Otx2 after IPAA treatment without injury. (14E-14H) Violin plots of markers used to define clusters, and bar plots showing cluster composition of IPA-IPA:Atoh1 (14E-14F) and Ascl1:Atoh1-IPA:Atoh1 (14G-14H) integrated scRNAseq data.

FIG. 15. Promotion of RGC production using transcription factors specific to developing retinal cells. Lentiviruses were used to induce the expression of several transcription factors that are expressed in developing retinal cells, but not in Müller glia. The Müller glia were grown in cell culture and infected with the viruses. The cells were then cultured for 5-7 days and subsequently processed for single cell RNAseq to determine their fates. Of all the factors tested, Onecut1 (arrow) was able to induce the Müller glia to generate new cells with the characteristic gene expression of RGCs.

FIG. 16. Promotion of RGC production using transcription factors specific to developing retinal cells. Onecut1 expression (as in FIG. 15) induced Müller glia to generate new cells with the characteristic gene expression of RGCs, including Nefm, Nefl, Ebf1 and Gap43.

FIGS. 17A-17C. AAV delivered reprogramming transcription factors can induce neurogenesis. HuC/D+ neurons lineage was traced from MG by tdTomato. (17A) AAV design using Atoh1. (17B) Protocol for lineage tracing. (17C) Immunofluorescent demonstration of successful delivery of reprogramming transcription factors resulting in HuC/D+ neurons reprogrammed from MG cells.

FIG. 18. Reprogramming efficiency of lineage-traced MG. Bar graph showing that both Atoh1 and Atoh7 delivered by AAV can induce HuC/D+ neurons. Atoh1 and Atoh7 are about the same in efficiency. AAV delivery of reprogramming factors under these conditions was much less efficient than transgenic expression.

FIG. 19. Irx2 and Neurod2 promote axon growth in IPA reprogrammed MG.

FIGS. 20A-20D. Human Muller glia generated in vitro from fetal retina or pluripotent stem cells using retinospheres and retinal organoids. (20A) Schematic of Embryonic Stem cell (ESC) differentiation protocol to generate retinal organoids (RO). (20B) Images of MG development in retinal organoids over time labeled with RLBP1 and SOX2. (20 C) Upper panel shows the schematic protocol for the generation of retinospheres (RS), lower panel shows images of retinospheres made from several fetal retinas and cultured for various times as labeled. (20D) Characterization of the MG in RS with RLBP1, VSX2, SOX9, SOX2 and GFAP.

FIGS. 21A-21B. Protocol to generate dissociated human Muller glia cultures in vitro.

FIGS. 22A-22E. Ascl1 promotes neurogenesis in human Muller glia. (22A) Protocol for delivery of GFP into dissociated cells. (22B-22E) These neurons express some pan neuronal markers such as DCX and TUJ1, but are not mature.

FIG. 23. Characterization of ShH10 capsid and RLBP promoter in NHP dissociated Muller glia culture.

FIG. 24. HES1 promoter as an alternative to RLBP1 promoter. Schematic illustration of a lentiviral construct containing HES1 promoter driving the expression of EGFP.

FIGS. 25A-25D. Schematic illustration of the construction of a lentivirus containing the HES1 promoter driving the expression of EGFP (control) or driving the expression of the proneural factor ASCL1 and EGFP in Muller glia (25A). (25B-25D) Testing HES1 promoter for specificity of MG expression in human retinospheres (D132+77=D 209 at the infection date). GFP and HES1 are found in the same cells.

FIG. 26. HES1-promoter also directs expression in MG in adult NHP dissociated cultures.

### DETAILED DESCRIPTION

The molecules, compositions, and methods described herein are based on the surprising discovery that retinal ganglion-like cells can be regenerated in the damaged adult retina in vivo with targeted overexpression of developmental retinal ganglion cell transcription factors. As demonstrated herein, AAV vectors can deliver reprogramming transcription factors to Muller glia in vivo. While the rate of neurogenesis from AAV reprogrammed Muller glia is much less than that observed with transgenic reprogramming, other RGC transcription factors can increase specific RGC genes, and in some examples, can improve RGC axon growth. In addition, the studies described herein show that human Muller glia can be reprogrammed to generate neurons in dissociated cultures, and these human Muller glia can be derived from either retinal organoids or fetal human retina. This ability to reprogram Muller glia into specific types is particularly important for endogenous regeneration strategies because most blinding diseases are the result of deficits in a particular neuronal subtype. For example, glaucoma is primarily caused by the death of RGCs.

### Definitions

All scientific and technical terms used in this application have meanings commonly used in the art unless otherwise specified. As used in this application, the following words or phrases have the meanings specified.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the disclosure herein.

As used herein, “retinal neuron” refers to any of the five types of neurons in the retina: photoreceptors, bipolar cells, ganglion cells, horizontal cells, and amacrine cells. In some particular embodiments, the retinal neurons are bipolar neurons, amacrine, horizontal, and ganglion cells.

As used herein, the terms “nucleic acid sequence” or “polynucleotide” refers to nucleotides of any length which are deoxynucleotides (i.e. DNAs), or derivatives thereof; ribonucleotides (i.e. RNAs) or derivatives thereof; or peptide nucleic acids (PNAs) or derivatives thereof. The terms include, without limitation, single-stranded, double-stranded, or multi-stranded DNA or RNA, genomic DNA, cDNA, DNA-RNA hybrids, oligonucleotides (oligos), or other natural, synthetic, modified, mutated or non-natural forms of DNA or RNA.

MicroRNAs, or “miRNAs”, or “miRs”, are short, non-coding RNAs that regulate gene expression by post-transcriptional regulation of target genes.

“Short hairpin RNAs” or “shRNAs” are synthetic or non-natural RNA molecules. shRNA refers to RNA with a tight hairpin turn used to silence (via RNA interference or RNAi) target gene expression in a cell. An shRNA is typically delivered via an expression vector such as a DNA plasmid or via viral vectors.

The term “vector” refers to, without limitation, a recombinant genetic construct or plasmid or expression construct or expression vector that retains the ability to infect and transduce non-dividing and/or slowly-dividing cells and integrate into the target cell's genome. The vector may be derived from or based on a wild-type virus. Aspects of this disclosure relate to an adeno-associated virus vector, an adenovirus vector, and a lentivirus vector.

The term “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be, without limitation, constitutive, inducible, repressible, or tissue-specific. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. An “enhancer” is a region of DNA that can be bound by activating proteins to increase the likelihood or frequency of transcription. Non-limiting exemplary enhancers and posttranscriptional regulatory elements include the CMV enhancer and WPRE.

The term “multicistronic” or “polycistronic” or “bicistronic” or tricistronic” refers to mRNA with multiple, i.e., double or triple coding areas or exons, and as such will have the capability to express from mRNA two or more, or three or more, or four or more, etc., proteins from a single construct. Multicistronic vectors simultaneously express two or more separate proteins from the same mRNA. The two strategies most widely used for constructing multicistronic configurations are through the use of 1) an IRES or 2) a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or sequences encoding the self-cleaving peptides.

The term “substantially complementary,” when used to define either amino acid or nucleic acid sequences, means that a particular sequence, for example, an oligonucleotide sequence, is substantially complementary to the sequence referenced. As such, typically the sequences will be highly complementary to the “target” sequence, and will have no more than 1, 2, 3, 4, 5, 6, 7, 8, 9, or 10 base mismatches throughout the sequence. In many instances, it may be desirable for the sequences to be exact matches, i.e. be completely complementary to the sequence to which the nucleic acid specifically binds, and therefore have zero mismatches along the complementary stretch. As such, highly complementary sequences will typically bind quite specifically to the target sequence region and will therefore be highly efficient in reducing, and/or even inhibiting the biological activity of the target sequence.

Substantially complementary nucleic acid sequences will be greater than about 80 percent complementary (or ‘% exact-match’) to the corresponding target sequence to which the nucleic acid specifically binds, and will, more preferably be greater than about 85 percent complementary to the corresponding target sequence to which the nucleic acid specifically binds. In certain aspects, as described above, it will be desirable to have even more substantially complementary nucleic acid sequences for use in the practice of the invention, and in such instances, the nucleic acid sequences will be greater than about 90 percent complementary to the corresponding target sequence to which the nucleic acid specifically binds, and may in certain embodiments be greater than about 95 percent complementary to the corresponding target sequence to which the nucleic acid specifically binds, and even up to and including 96%, 97%, 98%, 99%, and even 100% exact match complementary to the target to which the designed nucleic acid specifically binds.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of disclosed herein.

Percent similarity or percent complementary of any of the disclosed sequences may be determined, for example, by comparing sequence information using the GAP computer program, version 6.0, available from the University of Wisconsin Genetics Computer Group (UWGCG). The GAP program utilizes the alignment method of Needleman and Wunsch (1970). Briefly, the GAP program defines similarity as the number of aligned symbols (i.e., nucleotides or amino acids) which are similar, divided by the total number of symbols in the shorter of the two sequences. The preferred default parameters for the GAP program include: (1) a unary comparison matrix (containing a value of 1 for identities and 0 for non-identities) for nucleotides, and the weighted comparison matrix of Gribskov and Burgess (1986), (2) a penalty of 3.0 for each gap and an additional 0.10 penalty for each symbol in each gap; and (3) no penalty for end gaps.

“Nucleotide sequence” refers to a heteropolymer of deoxyribonucleotides, ribonucleotides, or peptide-nucleic acid sequences that may be assembled from smaller fragments, isolated from larger fragments, or chemically synthesized de novo or partially synthesized by combining shorter oligonucleotide linkers, or from a series of oligonucleotides, to provide a sequence which is capable of specifically binding to a target molecule and acting as an antisense construct to alter, reduce, or inhibit the biological activity of the target.

As used herein, “directed against”, in the context of antisense oligonucleotides, means the antisense oligonucleotide binds to a target miRNA and blocks or suppresses activity of the target.

As used herein, the terms “protein”, “peptide”, and “polypeptide” refer to amino acid subunits, amino acid analogs, or peptidomimetics. The subunits may be linked by peptide bonds. In another aspect, the subunit may be linked by other bonds, e.g., ester, ether, etc. As used herein the term “amino acid” refers to either natural and/or unnatural or synthetic amino acids.

As used herein, the term “recombinant expression system” or “recombinant expression vector” refers to a genetic construct for the expression of certain genetic material formed by recombination.

The term “effective amount” or “therapeutically effective amount” or “prophylactically effective amount”, refer to an amount of an active agent described herein that is effective to provide the desired/intended result and/or biological activity. Thus, for example, in various embodiments, an effective amount of a composition described herein is an amount that is effective to result in regeneration of retinal neurons, and/or to improve or to ameliorate symptoms of and/or to treat retinal degenerative diseases.

When the disclosure herein relates to a small molecule, polypeptide, protein, polynucleotide, nucleic acid, oligonucleotide, antisense, or miRNA, an equivalent or a biologically equivalent of such is intended within the scope of this disclosure. As used herein, the term “biological equivalent thereof” is intended to be synonymous with “equivalent thereof” when referring to a reference small molecule, polypeptide, protein, polynucleotide, nucleic acid, oligonucleotide, antisense, or miRNA even those reference molecules having minimal homology while still maintaining desired structure or functionality. Unless specifically recited herein, it is contemplated that any nucleic acid, polynucleotide, oligonucleotide, antisense, miRNA, polypeptide, or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

In some embodiments disclosed herein, the polypeptide and/or polynucleotide sequences are provided herein for use in gene and protein transfer and expression techniques described below. Such sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polynucleotide or polypeptide sequences are provided as examples of particular embodiments. Modifications may be made to the amino acid sequences by using alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, “treating” or “treatment” of a retinal degenerative disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the compositions, combination therapy, nucleic acid molecules, and methods disclosed herein for inducing neurogenesis from MG and/or generating functional neurons from MG, beneficial or desired results can include one or more, but are not limited to, alleviation or amelioration of one or more symptoms of retinal degeneration, diminishment of extent of a retinal degenerative condition (including a retinal degenerative disease), stabilized (i.e., not worsening) state of a retinal degenerative condition (including disease), delay or slowing of a retinal degenerative condition (including disease), progression, amelioration or palliation of a retinal degenerative condition (including disease), states of and remission of (whether partial or total) retinal degeneration, whether detectable or undetectable.

As used herein, the term “isolated” means that a naturally occurring DNA fragment, DNA molecule, coding sequence, or oligonucleotide is removed from its natural environment, or is a synthetic molecule or cloned product. Preferably, the DNA fragment, DNA molecule, coding sequence, or oligonucleotide is purified, i.e., essentially free from any other DNA fragment, DNA molecule, coding sequence, or oligonucleotide and associated cellular products or other impurities.

The term “cell” as used herein refers to either a prokaryotic or eukaryotic cell, optionally obtained from a subject or a commercially available source. Cells treated, transfected, transformed, or otherwise in contact with compositions and/or nucleic acid molecules disclosed herein, include without limitation, cells of a human, non-human animal, mammal, or non-human mammal, including without limitation, cells of murine, canine, or non-human primate species. Cells treated, transfected, transformed, or otherwise in contact with compositions and/or nucleic acid molecules disclosed herein are, without limitation, retinal cells, Müller glia (MG), and/or retinal neuronal cells such as retinal neurons, bipolar neurons, amacrine cells, horizontal cells, ganglion cells and/or glia. The term “Müller glial” cells “or “Müller glia” or “MG” refer to cells which are found in the vertebrate retina and are support cells for neurons. MG are the most common type of glial cells in the retina. While MG cell bodies are located in the inner nuclear layer of the retina, MG span across the entire retina.

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects.

As used herein, “a” or “an” means at least one, unless clearly indicated otherwise.

As used herein, to “prevent” or “protect against” a condition or disease means to hinder, reduce or delay the onset or progression of the condition or disease.

The term “encode” as it is applied to nucleic acid sequences refers to a polynucleotide which is said to “encode” a polypeptide, an mRNA, or an effector RNA if, in its native state or when manipulated by methods well known to those skilled in the art, can be transcribed and/or translated to produce the effector RNA, the mRNA, or an mRNA that can for the polypeptide and/or a fragment thereof. The antisense strand is the complement of such a nucleic acid, and the encoding sequence can be deduced therefrom.

As used herein, the term “expression” or “gene expression” refers to the process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “functional” may be used to modify any molecule, biological, or cellular material to intend that it accomplishes a particular, specified effect.

As used herein, the term “combined therapy” refers to two or more compositions and/or nucleic acid molecules, delivered in combination, for example and without limitation, sequentially, concurrently, simultaneously, and/or step-wise, in order to achieve a therapeutic effect.

The term “about,” as used herein when referring to a measurable value such as an amount, level or concentration, for example and without limitation, is meant to encompass variations of 20%, 10%, 5%, 1%, 0.5%, or even 0.1% of the specified amount, or fold differences in levels of a quantifiable comparison with a standard or control or reference material, such as 1-fold, 2-fold, 3-fold, 4-fold . . . 10-fold, 100-fold, etc. of the specified level of comparison.

In some embodiments, enhancing expression levels of the two or more proneural bHLH transcription factors, endogenous and/or exogenous, refers to an increase in the amount of expressed as compared to a control sample or explant levels of endogenous and/or exogenous Ascl1, and/or Atoh1, and/or Atoh7 such as, without limitation, untreated, or Ascl1 expression alone. In some embodiments, neurogenesis is increased and/or the production of functional neurons is increased as compared to a control. In some embodiments, expression levels and/or functional neurons are increased about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 2.5 fold, about 3 fold, about 4 fold, about 5 fold, about 6 fold, about 7 fold, about 8 fold, about 9 fold, about 10 fold, about 20 fold, about 50 fold, about 100 fold, about 1000 fold, or about 10,000 fold relative to the control.

In some embodiments, the terms “reprogramming potentiator” or “reprogramming potentiating agent”, used herein interchangeably, refers to a small molecule, polypeptide, protein, polynucleotide, nucleic acid, oligonucleotide, antisense, miRNA, or an equivalent or a biologically equivalent thereof which assists in the process of stimulating and/or boosting neurogenesis from MG in a manner such that functional neurons from the MG are produced. In one embodiment, one or more reprogramming potentiators assist in the process of stimulating neurogenesis and producing functional neurons from MG by inhibiting the HDAC pathway. In another embodiment, one or more reprogramming potentiators assist in the process of stimulating neurogenesis and producing functional neurons from MG by inhibiting the Jak/STAT pathway. In another embodiment, one or more reprogramming potentiators assist in the process of stimulating neurogenesis and producing functional neurons from MG by inhibiting the HDAC+Jak/STAT pathways. In another embodiment, one or more reprogramming potentiators assist in the process of stimulating neurogenesis and producing functional neurons from MG by enhancing and/or increasing endogenous and/or exogenous Ascl1 expression levels. See also our previous work in WO2019/210320 and WO02020/223308, each of which is incorporated herein by reference in its entirety.

The terms “acceptable,” “effective,” or “sufficient” when used to describe the selection of any components, ranges, dose forms, etc. disclosed herein intend that said component, range, dose form, etc. is suitable for the disclosed purpose.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 or 12, sequentially numbered, are disclosed in the prior art. Non-limiting exemplary serotypes useful in the methods disclosed herein include any of the 11 or 12 serotypes, e.g., AAV2, AAV5, and AAV8, or engineered serotypes, e.g. AAV-SHH10 and AAV-7m8. The AAV structural particle is composed of 60 protein molecules made up of VP1, VP2, and VP3. Each particle contains approximately 5 VP1 proteins, 5 VP2 proteins and 50 VP3 proteins ordered into an icosahedral structure.

### Nucleic Acid Molecules and Compositions

Provided are compositions and/or nucleic acid molecules for retinal regeneration, the potentiation of retinal regeneration, stimulation of regeneration of retinal ganglion cells, restoration of vision, and for treatment of retinal degenerative disease, damage, or injury.

Such nucleic acid molecules may be delivered by viral or non-viral means. One example of viral delivery is adeno-associated virus (AAV). Other examples include retrovirus and lentivirus delivery. One example of a non-viral method of delivery is cell penetrating peptide (CPP). Polynucleotide constructs may also be modified, such as through chemical modification, to improve their stability and/or suitability for delivery. In some embodiments, the oligonucleotide is modified by locked nucleic acids and/or phosphorothioate linkages. In some embodiments, a delivery system is selected for improved bioavailability, such as PEGylated liposomes, lipidoids, or biodegradable polymers, as examples.

In some embodiments, the composition further comprises one or more additional potentiating or therapeutic agents, including, for example, reprogramming potentiating agents. In some embodiments, the composition is free of reprogramming potentiating agents. Optionally, a composition comprising one or more small molecule reprogramming potentiating agents can be administered sequentially or concurrently with the nucleic acid molecules disclosed herein. In another embodiment, one or more protein/peptide or miR-based reprogramming potentiators can be incorporated into the nucleic acid molecules disclosed herein. Such one or more reprogramming potentiators are selected from HDACi, STATi, Jak/STATi and RNAi-based Ascl1 activators. See also our previous work in WO2019/210320 and WO2020/223308, each of which is incorporated herein by reference in its entirety.

In one embodiment, the HDAC signaling pathway inhibitor is selected from the group consisting of peptidomimetics, small molecule inhibitors, oligonucleotides, peptides and proteins. Representative examples of small molecule HDACi include, but are not limited to, trichostatin A (TSA), Istodax™ also known as (Pro)/romidepsin, Beleodaq™, also known as (Pro)/belinostat, Farydak™, also known as (Pro)/panobinostat, and Zolinza™, also known as (Pro)/vorinostat, Quisinostat, Abexinostat, Givinostat, Resminostat, Phenylbutyrate, Valproic Acid, Depsipeptide, Entinostat, Mocetinostat, and Tubastatin A. Exemplary HDACi peptides are, without limitation, 16cyc-HxA, 16lin-HxA and 16KA.

In some embodiments, the inhibitor, mimic, activator, or antagomir is an oligonucleotide or a nucleotide sequence. The invention thus provides nucleotide constructs for use in the compositions or combined therapy or nucleic acid molecules and methods described herein.

The reprogramming potentiating agents, in some embodiments, are selected from one or more STAT signaling pathway inhibitors; and one or more Ascl activators such as, without limitation, miR-25 and/or miR-124; and one or more let-7 family inhibitors.

Optionally, provided herein is a composition comprising any one or more of the combined therapy of RNAi-based Ascl1 activators and/or HDACi+STATi, and/or a nucleic acid sequence encoding the developmental RGC transcription factors or a vector comprising the nucleic acid sequences disclosed herein, and a carrier. In some embodiments, the carrier is a pharmaceutically acceptable carrier.

An exemplary nucleic acid sequence encoding human Onecut1 can be found at NCBI Reference Sequence number NC_000015.10 (SEQ ID NO: 1). An exemplary nucleic acid sequence encoding human Pou4f2 can be found at NCBI Reference Sequence number NC_000004.12 (SEQ ID NO: 3). An exemplary nucleic acid sequence encoding human Islet1 can be found at NCBI Reference Sequence number NC_000005.10 (SEQ ID NO: 5). An exemplary nucleic acid sequence encoding human Irx2 can be found at NCBI Reference Sequence number NC_000005.10 (SEQ ID NO: 8). The mouse version of Irx2 used in the Examples herein can be found at NCBI Reference Sequence: NM_010574.4 (SEQ ID NO: 64; encoding the amino acid sequence of SEQ ID NO: 65). An exemplary nucleic acid sequence encoding human Irx5 can be found at NCBI Reference Sequence number NC_000016.10 (SEQ ID NO: 13). The mouse version of Irx5 used in the Examples herein can be found at NCBI Reference Sequence: NM_018826.2 (SEQ ID NO: 66; encoding the amino acid sequence of SEQ ID NO: 67). An exemplary nucleic acid sequence encoding Neurod2 can be found at NCBI Reference Sequence number NC_000017.11 (SEQ ID NO: 17). An exemplary nucleic acid sequence encoding human Ebf1 can be found at NCBI Reference Sequence number NC_000005.10 (SEQ ID NO: 19). The mouse version of Ebf1 used in the Examples herein can be found at NCBI Reference Sequence: NM_001290709.1 (SEQ ID NO: 68; encoding the amino acid sequence of SEQ ID NO: 69). An exemplary nucleic acid sequence encoding human Tcf3 can be found at NCBI Reference Sequence number NC_000019.10 (SEQ ID NO: 48). The mouse version of Tcf3 used in the Examples herein can be found at NCBI Reference Sequence: NM_001164147.2 (SEQ ID NO: 70; encoding the amino acid sequence of SEQ ID NO: 71). This mouse sequence can be used to guide the selection of a corresponding human or humanized Tcf3.

An exemplary nucleic acid sequence encoding human Ascl1 can be found at NCBI Reference Sequence number NG_008950.1 (SEQ ID NO: 50). In another embodiment disclosed herein is a nucleic acid sequence encoding a human Ascl1 amino acid sequence or portion thereof of UniProtKB/Swiss-Prot: P50553.2 (SEQ ID NO: 51). Ascl1 homologues, e.g., derived from species such as murine, canine, equine, are included herein, without limitation.

The Protein Atonal Homolog 1 (Atoh1) is a proneural member of the family of bHLH transcription factors. The protein activates a different E box than the Ascl1 gene. An exemplary nucleic acid sequence encoding human Atoh1 can be found at NCBI Reference Sequence number NM_005172.1 (SEQ ID NO: 52). In another embodiment disclosed herein is a nucleic acid sequence encoding a human Atoh1 amino acid sequence or portion thereof of NP_005163.1 (SEQ ID NO: 53). Atoh1 homologs, orthologs and/or paralogs, e.g., derived from species such as murine, canine, equine, are included herein, without limitation.

The Atoh7 family bHLH transcription factor 7 (Atoh7) gene encodes a proneural member of the basic helix-loop-helix (BHLH) family of transcription factors. An exemplary nucleic acid sequence encoding human Atoh7 can be found at NCBI Reference Sequence number NM_008553.4 (SEQ ID NO: 54). In another embodiment disclosed herein is a nucleic acid sequence encoding a human Atoh7 amino acid sequence or portion thereof of NP_660161.1 (SEQ ID NO: 55). Atoh7 homologs, orthologs, and/paralogs, e.g., derived from species such as murine, canine, equine, are included herein, without limitation. An exemplary nucleic acid sequence encoding Neurogenin-2 (also known as NEUROG2 and NGN-2) can be found at NCBI Reference Sequence number NM_024019. An exemplary nucleic acid sequence encoding Neurod1 can be found at NCBI Reference Sequence number KR709666.

Exemplary nucleic acid sequences of the Ascl1, Atoh1, and/or Atoh7 or other proneural bHLH transcription factor for use herein include, without limitation, portions thereof of the corresponding sequences of Ascl1, Atoh1, and/or Atoh7, for the purposes of configurating multicistronic, bicistronic, and/or tricistronic constructs, plasmids, and/or expression vectors. It is understood that portions of the sequences referenced herein can be selected for use, wherein the selected portions are sufficient to encode the recited transcription factor(s) and/or other elements.

Exemplary sources for the promoter sequence can be found in HES1 (SEQ ID NO: 56), RLBP1 (SEQ ID NO: 57), or GLAST (also known as SL1A3; SEQ ID NOs: 61-63; see NCBI Reference Sequence Number NM_004172).

### Viral Vectors

In some embodiments, the vector disclosed herein is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers.

In some embodiments, the vector disclosed herein is an AAV vector with low toxicity. In some embodiments, the AAV vector does not incorporate into the host genome, thereby having a low probability of causing insertional mutagenesis. In some embodiments, the AAV vector can encode a range of total polynucleotides from 4.5 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh64R1 vector, and a modified AAV.rh64R1 vector and any combinations or equivalents thereof.

In some embodiments, the vector disclosed herein is a lentiviral vector. In one embodiment, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art. In some embodiments, exemplary lentiviral vectors that may be used in relation to any of the herein described compositions, nucleic acid molecules and/or methods, and can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIV_{SM}) vector, a modified sooty mangabey simian immunodeficiency virus (SIV_{SM}) vector, a African green monkey simian immunodeficiency virus (SIV_{AGM}) vector, a modified African green monkey simian immunodeficiency virus (SIV_{AGM}) vector, a equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

In some embodiments of the compositions and/or nucleic acid molecules and/or methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and/or nucleic acid molecules and/or methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11, or AAV12, or the vector and/or components are derived from a synthetic AAV serotype, such as, without limitation, Anc80 AAV (an ancestor of AAV 1, 2, 6, 8 and 9). In some embodiments, the viral vector is replication incompetent. In some embodiments, the viral vector is isolated or recombinant (rAAV). In some embodiments, the viral vector is self-complementary (scAAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer.

In some embodiments, expression vector or viral vector disclosed herein is used to transfect, transform, or come in contact with a cell which is a eukaryotic cell. In some embodiments, the cell is an animal cell. In some embodiments, the cell is a mammalian cell. In some embodiments, the cell is a bovine, murine, feline, equine, porcine, canine, simian, or human cell. In particular embodiments, the cell is a retinal neuron or MG of an animal or mammal.

In some embodiments, a cell is a packaging cell or a producer cell for production of a viral particle.

In some embodiments, provided herein are viral particles comprising, consisting of, or consisting essentially of a vector comprising, consisting of, or consisting essentially of a polynucleotide sequence encoding a developmental RGC transcription factor and, optionally, an Ascl1 protein.

In general, methods of packaging genetic material such as RNA or DNA into one or more vectors is well known in the art. For example, the genetic material may be packaged using a packaging vector and cell lines and introduced via traditional recombinant methods.

In some embodiments, the packaging vector may include, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging plasmids comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter.

The retroviral packaging plasmid may consist of two retroviral helper DNA sequences encoded by plasmid-based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the above env genes or chimeric envelope genes encoding the cytoplasmic and transmembrane of the above env gene products and a monoclonal antibody directed against a specific surface molecule on a desired target cell. Similar vector-based systems may employ other vectors such as sleeping beauty vectors or transposon elements.

The resulting packaged expression systems may then be introduced via an appropriate route of administration, discussed in detail with respect to the method aspects disclosed herein.

### Pharmaceutical Compositions

Pharmaceutical compositions disclosed herein include one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for intraocular administration.

### Cells

In some embodiments of the compositions and/or nucleic acid molecules and/or methods of the disclosure, a cell of the disclosure is a retinal cell, such as a Müller glial (MG) cell, or a rod or cone photoreceptor cell. In some embodiments, the cell is a neuronal cell. In some embodiments, a neuronal cell of the disclosure is a neuron of the retina. In some embodiments, a neuron cell of the disclosure is a neuron of an optic nerve. In some embodiments, a neuron cell of the disclosure is a neuroglial or a glial cell. In some embodiments, a cell is a bipolar neuron, a horizontal cell, a ganglion cell, or an amacrine cell. In some embodiments, a cell of the disclosure is an astrocyte. In some embodiments, cells of the disclosure are macroglia or microglia or glia.

In some embodiments of the compositions and methods of the disclosure, a cell of the disclosure is a cultured cell.

In some embodiments of the disclosure, a cell is in vivo, in vitro, ex vivo, or in situ. In some embodiments, the cells are modified ex vivo and transplanted into and/or administered to the retina of a subject in need thereof.

In some embodiments, a cell of the disclosure is autologous or allogeneic and used for transplantation.

In some embodiments, a cell of the disclosure is a stem cell-derived or an embryonic stem cell-derived retinal cell. In some embodiments, the cell is derived from an induced pluripotent stem cell (iPS cell)-derived retinal cell. In some embodiments, the cell is derived from a retinal organoid.

### Methods

Described herein are methods for stimulating regeneration of retinal ganglion cells, and/or inducing retinal regeneration, in a subject. Also provided are methods for enhancing retinal regeneration, improving retinal neurogenesis, potentiating retinal regeneration, restoring vision, and treating retinal degenerative disease, damage, injury, or blindness.

Also provided herein are methods for inducing retinal regeneration comprising administering to a subject a composition as described herein. In some embodiments, the methods are effective to increase the number of Müller glial-derived retinal ganglion cells, to induce Müller glial (MG) cells to enter the mitotic cell cycle, and/or to generate new retinal neurons, including the generation of new ganglion cells. In some embodiments of the method, the number of retinal neurons increases by at least 25% relative to a baseline level or other reference amount representative of an untreated retina. In other embodiments, the number of retinal neurons increases by at least 40%. In some embodiments, the number of retinal neurons increases by 10%, 20%, 50%, 100%, 150%, 200%, or more.

Optionally, methods disclosed herein may utilize combined therapy compositions comprising one or more, or two or more, small molecule reprogramming potentiating agents. The agents can be administered sequentially or concurrently with the nucleic acid molecules disclosed herein. In another embodiment, one or more protein/peptide or miR-based reprogramming potentiators can be incorporated into the nucleic acid molecules used in the methods disclosed herein. In some embodiments, the method is performed in the absence of such reprogramming potentiators.

The subject is typically a mammal, such as a human or veterinary subject. In one embodiment, the subject is an adult. The subject, in some embodiments, has a retinal degenerative disease. Examples of such retinal degenerative diseases include, but are not limited to, Age-related Macular Degeneration (AMD), Retinitis Pigmentosa (RP), Diabetic Retinopathy (DR), Central Retinal Artery Occlusion (CRAO). Vitreoretinopathy, and Glaucoma.

### Administration and Dosage

The compositions and/or nucleic acid molecules disclosed herein are administered in any suitable manner, often with pharmaceutically acceptable carriers. Suitable methods of administering compositions, compounds, molecules, nucleic acids, and vectors in the context of the present invention to a subject's eye or retina are available, and, although more than one route can be used to administer a particular composition, a particular route can often provide a more immediate and more effective reaction than another route. For treatment of the retina, intraocular injection, such as, for example and without limitation, intravitreal injection and subretinal injection are the most common routes of delivery to the retina. In some embodiments, however, periocular, suprachoroidal, systemic, or topical administration is more suitable for efficacy and safety of delivery.

The dose administered to a patient, in the context of the disclosure herein, should be sufficient to result in a beneficial therapeutic response in the patient over time, or to inhibit disease progression. Thus, the composition is administered to a subject in an amount sufficient to elicit an effective response and/or to alleviate, reduce, cure or at least partially arrest symptoms and/or complications from the retinal disease or injury. An amount adequate to accomplish this is defined as a “therapeutically effective dose.”

Routes, order and/or frequency of administration of the therapeutic compositions disclosed herein, as well as dosage, will vary from individual to individual, and may be readily established using standard techniques. In general, an appropriate dosage and treatment regimen provides the active compound(s) in an amount sufficient to provide therapeutic and/or prophylactic benefit. Such a response can be monitored by establishing an improved clinical outcome in treated patients as compared to non-treated patients.

### EXAMPLES

The following examples are presented to illustrate the present invention and to assist one of ordinary skill in making and using the same. The examples are not intended in any way to otherwise limit the scope of the invention.

### Example 1: Reprogramming Müller Glia to Regenerate Ganglion-Like Cells with Developmental Transcription Factors

Many neurodegenerative diseases cause degeneration of specific types of neurons. For example, glaucoma leads to death of retinal ganglion cells, leaving other neurons intact. Neurons are not regenerated in the adult mammalian central nervous system. However, in non-mammalian vertebrates, glial cells spontaneously reprogram into neural progenitors and replace neurons after injury. We have recently developed strategies to stimulate regeneration of functional neurons in the adult mouse retina by overexpressing the proneural factor Ascl1 in Müller glia. In this Example, we test additional transcription factors (TFs) for their ability to direct regeneration to particular types of retinal neurons. We engineered mice to express different combinations of TFs in MG, including Ascl1, Pou4f2, Islet1 and Atoh1. Using IHC, scRNA-seq, scATAC-seq, and electrophysiology we find retinal ganglion-like cells can be regenerated in the damaged adult mouse retina in vivo with targeted overexpression of developmental RGC-transcription factors.

In the past few years, our group and others have found strategies to stimulate MG in adult mice to behave similar to their fish counterparts and generate neurons (4-9). We screened a number of transcription factors (TFs) that were differentially expressed between mouse MG and retinal progenitors for their ability to stimulate neurogenesis in MG in vitro (10). One factor that emerged from this screen was the proneural basic helix-loop-helix (bHLH) TF Ascl1, which was able to induce neurogenesis from MG in vivo in young mice (11) and when cotreated with a histone deacetylase [trichostatin A (TSA)] in adult mice (4). Ascl1-expressing MG adopt a molecular phenotype similar to developing retinal progenitors, and a subset of these cells undergoes mitotic division (5, 12). Some of these newly generated cells go on to differentiate into retinal neurons that connect with the endogenous circuitry (4, 5). Using Ascl1 to stimulate MG neurogenesis causes most MG-derived neurons to take on a bipolar cell fate, with a minority resembling amacrine cells (4, 12). Recently, we reported that the efficiency and range of neuronal cell types generated through MG reprogramming can be substantially improved by adding an additional bHLH TF of the atonal class (Atoh1/7). With this combination, up to 80% of the MG expressing Ascl1 and Atoh1 will become neurogenic precursors and ultimately neurons (13).

Our previous results show that combining two TFs of the same type (bHLH proneural) causes a substantial increase in the efficiency of in vivo MG reprogramming, but we were unable to control the types of neurons generated by MG in this paradigm. This is in some way similar to the process of regeneration in zebrafish, where injury triggers the generation of all neuronal types, regardless of whether the injury is widespread or targeted to specific cell types (14, 15). However, particular retinal diseases often are the result of a defect in a specific neural class. For example, in glaucoma, blindness results from the loss of retinal ganglion cells (RGCs) (16). Therefore, an impetus exists to direct endogenous regeneration to a particular affected type of neuron for cell replacement strategies.

Here, we test whether combining Ascl1 with other types of developmentally important TFs can more precisely direct MG-derived retinal progenitors to specific retinal cell fates. Specifically, we explore the effects of combining Ascl1 overexpression with two other TFs of different classes, Islet1, a LIN, Islet1, MEC3 (LIM) homeodomain TF, and Pou4f2, a class IV, Pit-Oct-Unc (POU) homeodomain TF. Both factors have been shown to be important for cell fate determination in the developing retina, with Pou4f2 necessary for RGC differentiation and Islet1 important in the development of several types of retinal neurons including RGCs (17, 18).

We find that the expression of Pou4f2 and Islet1, along with Ascl1, in adult mouse MG, directs a subset of the neurons generated by the MG toward a cell fate that resembles RGCs. The MG-derived RGC-like neurons (i) can be immunolabeled with markers of normal RGCs; (ii) have a transcriptome similar to developing RGCs by single-cell RNA sequencing (scRNA-seq); (iii) have a broader range of electrophysiological characteristics than neurons generated by Ascl1 alone, such as action potentials; and (iv) display a pattern of chromatin accessibility similar to developing RGCs. Together, our results show that neural regeneration from MG can be directed to specific cell types using combinations of developmentally relevant TFs.

### Materials and Methods

### Primary Cell Culture

Retinas from tetO-Ascl1-GFP or tetO-Pou4f2-Islet1-Ascl1-GFP mice of both sexes were harvested at Postnatal Day 12 (P12) for MG cultures. For dissociation, the retinas were incubated in a solution of papain and deoxyribonuclease (DNase) (Worthington) for 10 min at 37° C., followed by trituration. To stop the reaction, an equal volume of ovomucoid (Worthington) was added. Cells were then spun at 4° C. at 300 g for 10 min and resuspended in growth medium consisting of Neurobasal (Gibco), 10% fetal bovine serum (FBS) (Clontech), N2 (Invitrogen), 1 mM L-glutamine (Invitrogen), 1% penicillin-streptomycin (Invitrogen), and mouse epidermal growth factor (100 ng/ml) (R&D Systems). Cells were plated at a density of two retinas per 10 cm2 in a six-well dish and incubated at 37° C. The medium was changed every 2 days until confluent (˜7 days). At confluence, cells were passaged with TrypLE (Gibco); resuspended in a freezing medium consisting of 50% growth medium, 40% FBS, and 10% dimethyl sulfoxide (DMSO); and stored at least 1 day in liquid N02 for at least 24 hours. After thawing, cells were grown in FBS-reduced growth medium (1%) and treated with doxycycline to induce tetO-mediate genes.

### Animals

All animals were treated and housed with University of Washington Institutional Animal Care and Use Committee approved protocols. The (i) Glast-CreER:LNL-tTA:tetO-mAscl1-ires-GFP, (ii) Glast-CreER:LNL-tTA:tetO-P&I:tetO-mAscl1-ires-GFP, (iii) Glast-CreER:LNL-tTA:tetO-Atoh1:tetO-P&I:tetO-mAscl1-ires-GFP mice, (iv) rtTa:tetO-Ascl1-ires-GFP, and (v) rtTa:tetO-P7I:tetO-Ascl-ires-GFP are from mixed backgrounds of C57BL/6 and B6SJF1. The Glast-CreER, LNLtTA, and rtTa mice are from the Jackson Laboratory. The tetO-mAscl1-GFP mice were a gift from M. Nakafuku (University of Cincinnati), the tetO-Atoh1 mice were a gift from P. Chen (Emory University), and the tetO-P&I mice were a gift from X Mu (University of Buffalo). Males and females were both used in experiments at equal frequencies. All in vivo experiments were performed on adult mice that were over 40 days old.

### Immunohistochemistry

After CO2-mediated euthanasia, corneas were removed and eye globes were fixed for 30 min in 4% paraformaldehyde (PFA) in phosphate-buffered saline (PBS). Fixed eyes were then incubated overnight in 30% sucrose at 4° C. Retinas were then frozen in optimal cutting temperature compound (O.T.C.) and cryosectioned at 18 μm. For immunostaining, sections were washed two times for 10 min in PBS and then incubated in primary antibody in 0.5% Triton X-10 and PBS overnight. Slides were then washed in PBS and incubated in secondary antibodies for 1 hour. Last, slides were washed again in PBS and coverslipped with Fluoromount-G (SouthernBiotech). See Table 1 for antibody and concentration information.

Cultured MG were plated on glass coverslips with FBS-reduced growth medium (described above). To activate the transgenes, doxycycline was added at a concentration of 3 μg/ml every 24 hours for a period of 5 days. The coverslips were washed with PBS and then fixed with 4% PFA for 10 min at room temperature (RT), followed by 3×5 min of washes with PBS. Primary antibody incubation was done in 0.5% Triton X-10 and 10% normal horse serum (NHS) in PBS overnight at 4° C. Coverslips were washed in PBS and then click chemistry was used to label EdU (Click-iT EdU Assay, Invitrogen), followed by incubation with secondary antibodies and 4′,6-diamidino-2-phenylindole at RT for 1 hour. After staining, the coverslips were washed three more times and placed onto slides with Fluoromount-G (SouthernBiotech).

TABLE 1
List of antibodies used in the study. Columns identify the antibody, source, catalog
number, RRID identifier and concentration used for immunohistochemistry.

| | | | |
|---|---|---|---|
| REAGENT or | | | |
| RESOURCE | SOURCE | IDENTIFIER | CONCENTRATION |

Antibodies

| | | | |
|---|---|---|---|
| Goat anti-Brn3 | Santa Cruz | Cat#: SC-6026 | 1:300 |
| | | RRID: AB_673441 | |
| Rabbit anti-Calbindin | Millipore | Cat#: AB1778 | 1:1000 |
| | | RRID: AB_2068336 | |
| Rabbit anti-Calretinin | SW Ant | Cat#: 7699 | 1:500 |
| | | RRID: AB_10000321 | |
| Chicken anti-GFP | Abcam | Cat#: Ab13970 | 1:1000 |
| | | RRID: AB_300798 | |
| Mouse anti-HUC/D | Invitrogen | Cat#: A-21271 | 1:500 |
| | | RRID: AB_221448 | |
| Mouse anti-Islet1 | DSHB | Cat#: 40.2D6 | 1:50 |
| | | RRID: AB_528315 | |
| Mouse anti-Neu/N | Millipore | Cat#: MAB377 | 1:1000 |
| | | RRID: AB_2298767 | |
| Rabbit anti- | Chemicon | Cat#: AB1982 | 1:500 |
| Neurofilament | | RRID: AB_2313731 | |
| Goat anti-OTX2 | R&D Systems | Cat#: BAF1979 | 1:250 |
| | | RRID: AB_2157171 | |
| Rabbit anti-SATB1 | Abcam | Cat#: Ab109122 | 1:300 |
| | | RRID: AB_10862207 | |
| Goat anti-Sox2 | Santa Cruz | Cat#: SC-17320 | 1:1000 |
| | | RRID: AB_2286684 | |
| Rabbit anti-Tuj1 | Covance | Cat#: MRB-435p | 1:500 |
| | | RRID: AB_2313773 | |

Secondaries

| | | | |
|---|---|---|---|
| Donkey anti-chicken 488 | Jackson Immuno | Cat#: 703-545-155 | 1:1000 |
| Donkey anti-goat 568 | Life Technologies | Cat#: A11057 | 1:1000 |
| Donkey anti-goat 647 | Jackson Immuno | Cat#: 705-605-147 | 1:1000 |
| Donkey anti-mouse 568 | Life Technologies | Cat#: A10037 | 1:1000 |
| Donkey anti-mouse 647 | Jackson Immuno | Cat#: 715-605-150 | 1:1000 |
| Donkey anti-rabbit 568 | Life Technologies | Cat#: A100042 | 1:1000 |
| Donkey anti-rabbit 647 | Thermo Fisher | Cat#: A-31573 | 1:1000 |
| | Scientific | | |

### Fluorescence-Activated Cell Sorting

Following euthanasia, retinas were dissociated into single cells as described for cell culture; after pelleting at 300 g at 4° C., cells resuspended in Neurobasal solution and passed through a 35-μm filter. Using a BD FACSAria III cell sorter (BD Bioscience), FACS was performed on GFP+ cells.

### Injections

Intravitreal injections were performed with a 32-G Hamilton syringe on mice anesthetized with isoflurane. Injections of NMDA were done in a volume of 1 μl at a concentration of 100 mM in PBS. TSA (Sigma-Aldrich) was administered via intravitreal injections in DMSO at a concentration of 1 μg/μl. Intraperitoneal injections of tamoxifen (1.5 mg per 100 μl of corn oil) were administered to adult mice for four consecutive days to induce expression of the tetO-mAscl1-ires-GFP, the tetO-P&I, and the tetO-Atoh1 gene.

### Microscopy/Cell Counts

Images were taken on a Zeiss LSM880 confocal microscope. For quantification of cell counts, a minimum of four images per retina with a 20× objective were taken at the same magnification.

### Electrophysiology

Recordings were performed identical to our previous reports (4, 5). Mice were dark-adapted before recordings. After euthanasia, retinas were sliced into 200-μm slices for recording. Tissue recordings were performed in Ames medium at 32° C. and oxygenated with 95% O₂/5% CO2. GFP+ cells were targeted for recording using video differential interference contrast with infrared light and confocal microscopy. Light responses were measured under infrared conditions, and the tissue was exposed to full-field illumination via blue and green light-emitting diodes. Recordings were performed using pulled glass pipettes and filled with solution containing the following: 123 mM K-aspartate, 10 mM Hepes, 1 mM MgCl2, 10 mM KCl, 1 mM CaCl2, 2 mM EGTA, 0.5 mM tris-guanosine triphosphate, 4 mM MG-adenosine triphosphate, and 0.1 mM Alexa Fluor 695 hydrazide.

### Single-Cell RNA Library Construction

For in vivo datasets, following FACS purification of GFP+MG, cells were centrifuged at 300 g at 4° C. and resuspended at a concentration of 1000 cells/μl. Library construction was done using 10× Genomics 3′ single-cell RNA V3 or V3.1 sequencing kits as described by the manufacturer. Cells were encapsulated in gel beads and in given unique barcodes using the 10× Chromium controller and 10× Genomics chip type G. Libraries were multiplexed using single and dual index kits (10× Genomics).

For in vitro experiments, MG cultures from three to five mice were used. Following 5 days of doxycycline treatment, cells were dissociated using a mix of Accutase (Sigma-Aldrich, Saint-Louis, MO) and DNAse (Worthington, Lakewood, NJ) for 2 min and then spun down at 400 relative centrifugal force for 7 min at 4° C. The pellet was resuspended in culture medium to reach a targeted concentration of 1000 cells/μl. Cells were passed through a strainer and loaded into the 10× Genomics Chromium Single Cell chip G following the protocol of Chromium Single Cell 3′ Reagents Kits v3.1 (10× Genomics, Pleasanton, CA).

### Single-Cell RNA Sequencing, Mapping, and Data Analysis

Libraries were sequenced using an Illumina NextSeq 500, in most cases using multiplexed libraries using high-output 150 kits. Data were demultiplexed and aligned to the mm10 genome using CellRanger version 3.0. Filtered output files were further analyzed in R using Seurat version ≥3.0, ggplot2, data.table, dplyr, tidyr, and other commonly used R packages. Low-quality cells (identified as having low read depth or high mitochondrial content; >10%) were removed from datasets. Microglia, as well as any astrocytes or endothelial cells present (for in vitro experiments), were identified and removed from the data before downstream analysis. In addition, before analysis, gene expression data were normalized and scaled, and cells were clustered using principal components analysis and UMAP, using the tools available in the Seurat R package version ≥3.0. Comparisons between datasets were done by canonical correlation analysis as described by the Satija laboratory vignette (46) (https://satijalab.org/seurat/archive/v3.0/integration.html).

Integration with Development Data

For comparison to developing retina, data were first downloaded from Gene Expression Omnibus (GEO) (21). The label transfer was carried out in Seurat using a reference dataset composed of 432 randomly sampled cells of each major cell class in the developing retina dataset (Rod, Cone, Bipolar, Amacrine, Horizontal, Retinal Ganglion, and Müller glia), for a total of 3024 cells, as identified by canonical markers. Reads were downsampled to a common average depth before analysis. In addition, the IPA neurons were integrated directly with a subset composed only of E14 cells from the development dataset.

### Single-Cell ATAC Sequencing

The Cellranger ATAC pipeline (2.0.0) was used to preprocess the data resulting from sequencing (47). First, “cellranger-atac mkfastq” was used to convert BCL files to fastqs and demultiplex reads. Next, “cellranger-atac count” was run to map Tn5 sites to mm10 (mouse genome), remove duplicate reads, and remove background cells. This returned peak by cell matrices and barcoded fragment files that were loaded into Signac (48), an R (4.0.4) (R core team, 2021) package. Macs2 was then run on the Signac object and barcoded fragment files to call peaks using Signac's “CallPeaks” function (49). Fragments were mapped to the peaks called by Macs2 and assigned to cells using Signac's “FeatureMatrix” function. Further quality control (QC) metrics were measured in Signac using the “NucleosomeSignal” and “TSSEnrichment” functions. Cells who were outliers in the QC metric categories were removed as per Signac's standard processing guidelines. Latent semantic indexing (LSI) was performed in Signac using the “RunTFIDF” and “RunSVD” functions. Signac's “DepthCor” was used to identify LSI dimensions that were highly correlated with read depth; these LSI dimensions were excluded from downstream analysis. Signac/Seurat's “RunUMAP” function was run to compute the UMAP embedding. To identify clusters, Signac/Seurat's “FindClusters” was then run at varying resolutions. Clusters were assigned to known retinal cell types by inspecting Tn5 insertions within 100 kb of known marker genes using Signac's “CoveragePlot” and further supported using chromVAR scores for known lineage-specific TFs. Clusters of the same type were grouped for visualization purposes. Vertebrate motifs were acquired from the Jaspar 2020 database. Signac's “AddMotifs” function was used to map these motifs to peaks within the Signac object. Signac's “RunChromvar” function was used to calculate motif accessibility z score across all cells.

### Dataset Integration

Before integrating Signac objects, we first ran all previous computational steps on each sample independently. Next, we created a shared peak set for all objects that were to be integrated using BEDOPS (-m) (50). Signac's FeatureMatrix function was run on each sample with the merged peak set to put all samples in the same feature space. Samples were next downsampled to the same average read depth using DropletUtils “downsampleMatrix” function. Samples were merged using Signac/Seurat's “merge” function, and standard Signac normalization, dimensional reduction, clustering, and visualization were performed on the merged object as described above. The object was then split by samples, and the samples were integrated together. Anchors between samples were calculated using “FindIntegrationAnchors,” and an integrated embedding space was then calculated using Signac's “IntegrateEmbeddings” function. UMAP and clustering for the integrated object were performed as previously described.

### Pseudotime Analysis

To calculate pseudotime and identify trajectory branches, samples were loaded into Monocle 3 (51) using SeuratWrapper's “as.cell_data_set” function. Clustering was performed and partitions were defined using Monocle 3's “cluster_cells.” Monocle 3's “learn_graph” was then run to define the principal trajectory graph. The pseudotime root was placed in the progenitor-like or MG-like cluster using Monocle 3's “order_cells.” Branches were selected using Monocle 3's “choose_graph_segments.” Pseudotime and branch data were transferred back to the Seurat object for later analysis.

### Scatterplots

To create the scatterplots, Signac/Seurat's “FindMarkers” function was run between cell groups of interest, using the top 25% most accessible peaks in those cell groups. A scatterplot was then made showing the percent of cells in each group that had accessibility of each peak. Peaks with an average log base twofold change greater than 0.05 were selected for each group. For GO analysis, these peak sets were then loaded into GREAT (Genome Regions Enrichment of Annotations Tool) (52) or analyzed with gprofiler2 package in R for scRNA-seq data.

### Cascade Heatmaps

To construct the Cascade heatmaps, motif names were translated to mouse gene names using R and biomart's “getLDS” function. Pseudotime lineage branches for the RGCs were subset from the RNA and ATAC data from E14 samples. Variable motifs were identified by running chromVAR's “addGCBias,” “getBackgroundPeaks,” “computeDeviations,” and “computeVariability” functions. Peaks with a verbality score greater than 1.2 were kept for further analysis. This motif list was then subset again; only motifs corresponding to TFs expressed by more than 10% of cells in the RGC branch were kept. Last, conjoined motifs were dropped. Motif enrichment scores for selected motifs were ordered over pseudotime in the E14 RGC branch in the scATAC-seq data by fitting their ChomVAR scores to a third-order polynomial function and ordering motifs by the maximum value of this function within our pseudotime range. To create the heatmaps, chromVAR motif accessibility z scores were plotted in the previously derived order over pseudotime within the RGC lineage. RNA heatmaps were created by plotting the genes whose binding sites were in the final motif list in the same order across pseudotime in the RNA object within RGC lineage. The cascade heatmap for the reprogrammed cells was created by plotting motifs and factors identified in the developmental data.

### Results

Pou4f2 and Islet1 increase the diversity of neurons from Ascl1-reprogrammed MG

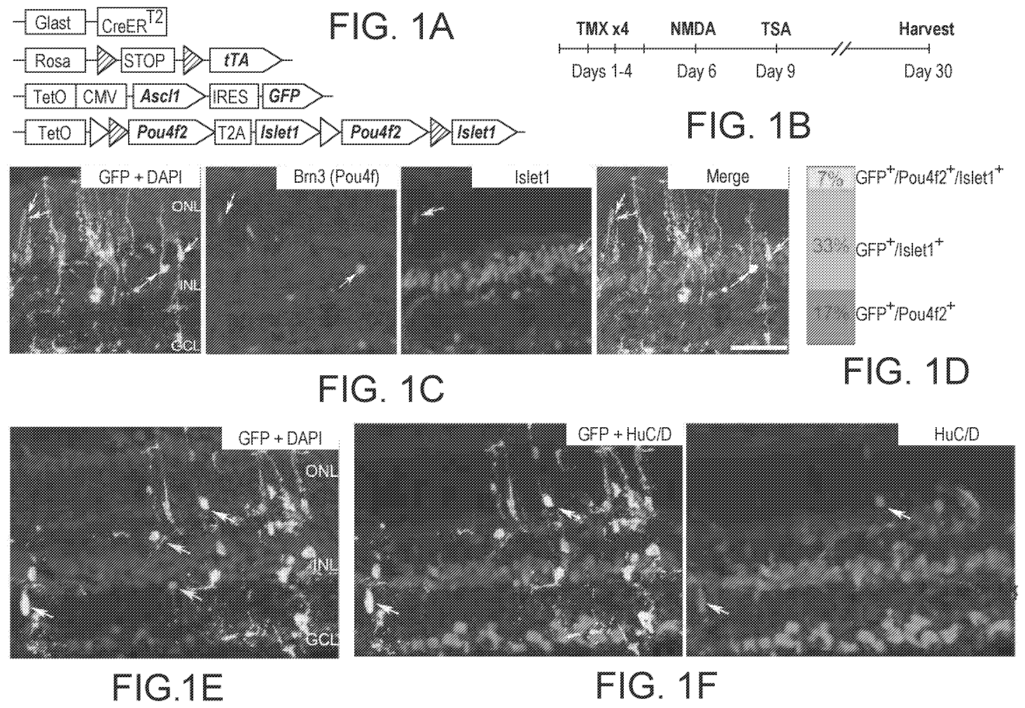

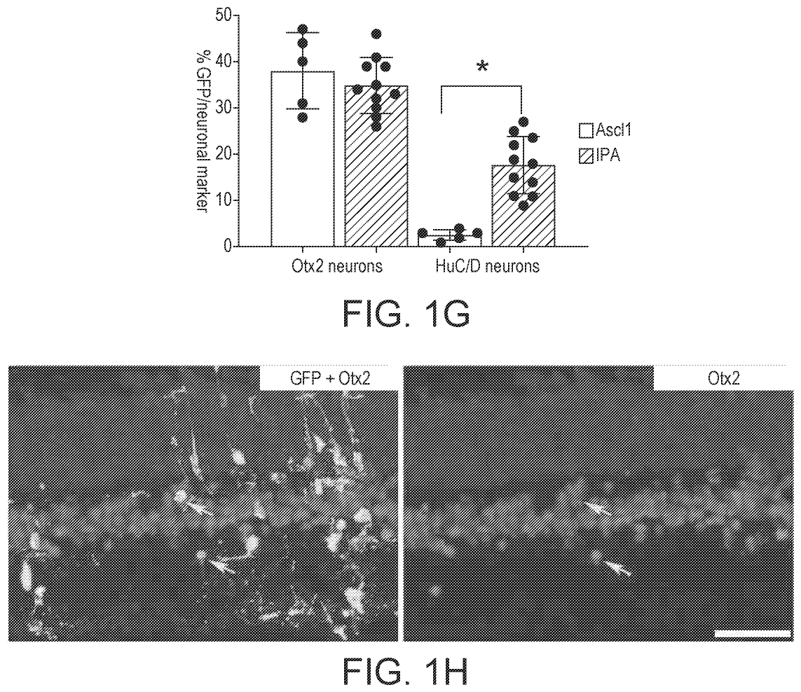

We previously developed a mouse where Ascl1 is induced specifically in MG (Glast-CreER:LNL-tTA:teto-mAscl1-GFP:ccGFP) by application of tamoxifen. After Ascd1 induction, retinal injury [N-methyl-D-aspartate (NMDA)] followed by injection of a histone deacetylase inhibitor (TSA) induces MG to express genes associated with developing retinal progenitors and to generate new neurons in the adult mouse retina in vivo. Most of these newly generated neurons adopt a bipolar cell fate (4, 5, 12). To examine whether expression of additional TFs (Pou4f2 or Islet1) can direct the MG-derived progenitors to other neuronal fates, we crossed the Glast-CreER:LNL-tTA:teto-mAscl1-GFP mice with a tetO-Pou4f2-tetO-Islet1 transgenic mouse line (tetO-IPA; FIG. 1A). This construct encodes Pou4f2 and Islet1 separated by two different loxP variants (19). When exposed to Cre recombinase, this cassette allows for expression of either Pou4f2, Islet1, or sometimes both.

The tetO-IPA mouse line allows us to test whether MG reprogramming is enhanced by treatment with Pou4f2+Ascl1, Islet1+Ascl1, or Islet1+Pou4f2+Ascl1 (hereafter IPA). After intraperitoneal application of tamoxifen to induce the TFs in MG, we induced a retinal injury by intravitreal injection of NMDA, followed by TSA (FIG. 1B). The mice were then euthanized 3 weeks later for immunofluorescence analysis to assess the fate of recombined MG (FIG. 1, C to E). FIG. 1 (C and D) shows that this protocol induces the transgenes in MG and neurons derived from them [green fluorescent protein positive (GFP+)]. We found GFP+MG-derived cells expressing either Brn3 (Pou4f2) (17%), Islet1 (33%), or both TFs (7%) (FIG. 1, C and D).

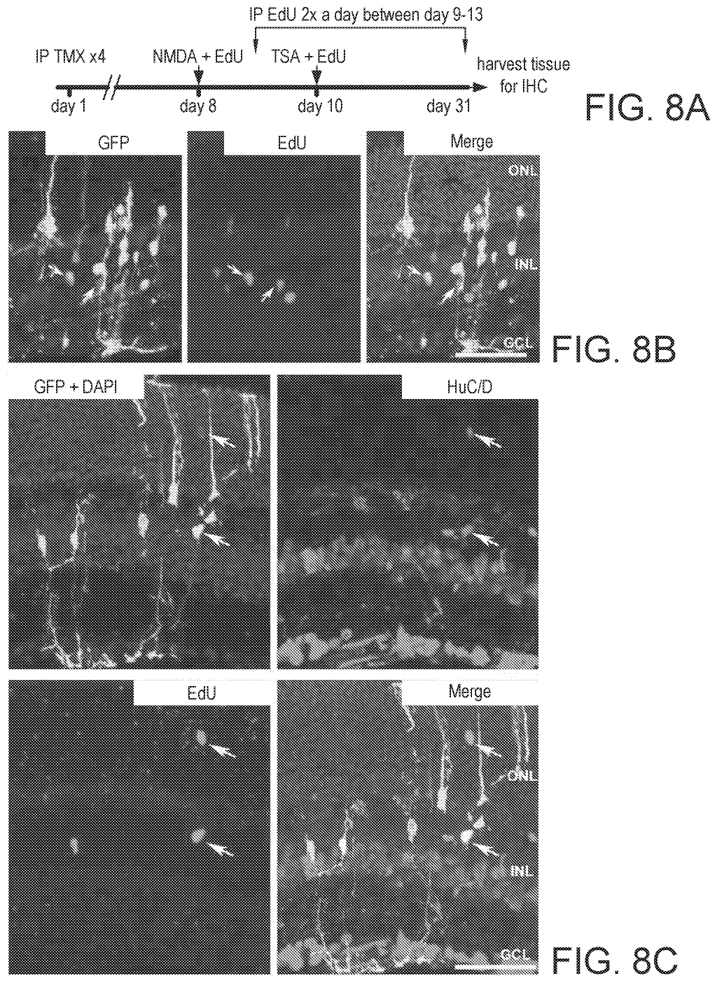

Additional immunohistochemistry (IHC) analysis demonstrated that the IPA combination effectively promoted neurogenesis from MG. Most glial-derived cells acquire a neuronal morphology 3 weeks after injury (FIG. 1E). Quantifications of MG-derived cells in retinal sections from IPA-treated mice confirmed that the MG-derived GFP+ neuronal-like cells expressed the ganglion/amacrine marker HuC/D (FIG. 1, F and G) or the bipolar marker Otx2 (FIG. 1, G and H). IPA expression substantially enhanced MG neurogenesis of HuC/D neurons compared to Ascl1 alone (FIG. 1G). Consistent with our previous reports, a subset of the MG-derived neurons was derived from EdU+, proliferating MG (FIG. 8A-8C). Together, these data suggest that the addition of TFs Pou4f2 and/or Islet1 enhances the neurogenic capacity and expands the resulting cell fates of Ascl1-MG in vivo.

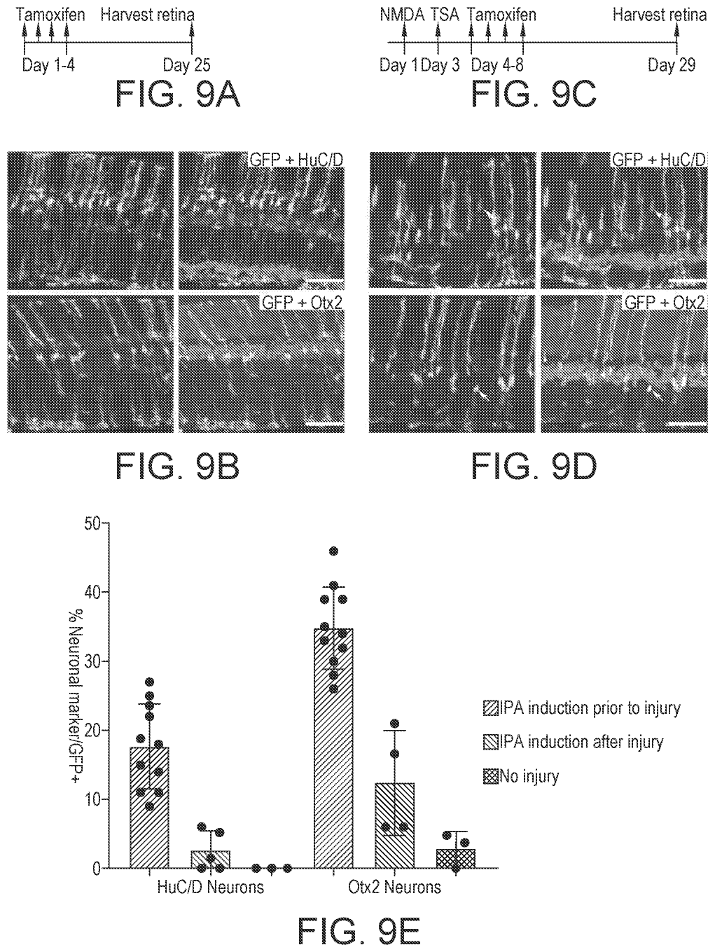

We had previously found that the combination of Ascl1:Atoh1 could stimulate neurogenesis from MG in the absence of retinal damage (13). In contrast, IPA induction in the undamaged retina was not sufficient to stimulate neurogenesis (FIG. 9, A, B, and E). We also tested whether MG could undergo neurogenic reprogramming if we activated the IPA factors in MG after NMDA injury and TSA rather than before NMDA and TSA as shown in FIG. 1. We found that induction of IPA after injury stimulated a small amount of neurogenesis, but substantially less than our original paradigm (FIG. 9, C to E).

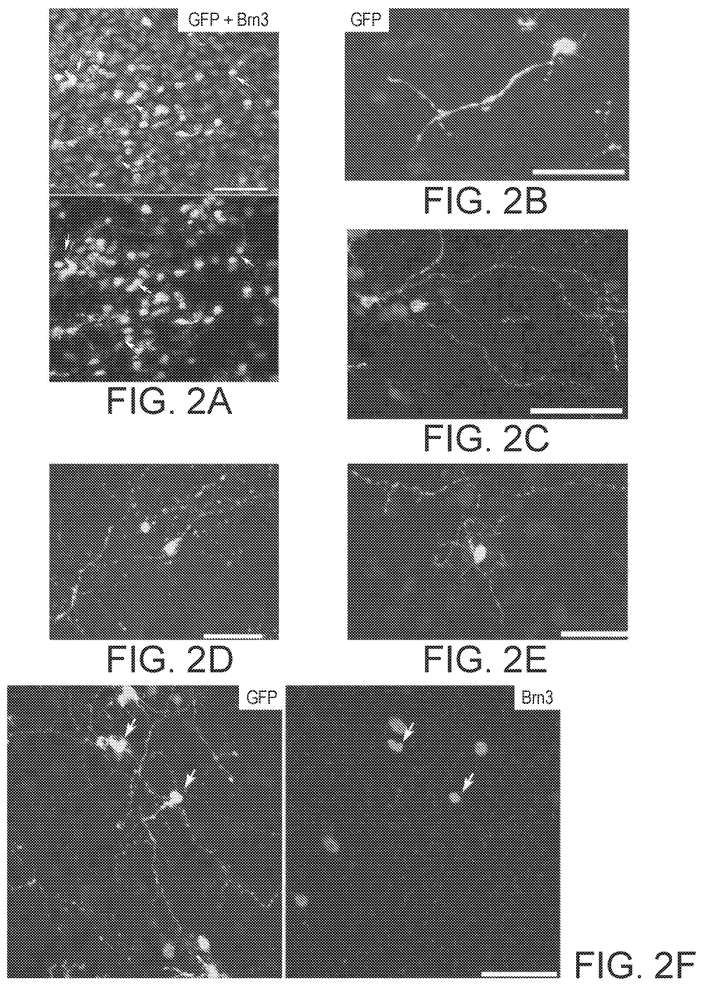

Because a substantial portion of the newly generated neurons in the damaged retina expressed the ganglion/amacrine marker HuC/D, we performed whole-mount imaging of IPA-derived neurons to better assess their neuronal morphology. This revealed cells with large branching dendritic arbors reminiscent of RGCs or wide-field amacrine cells; cells with this morphology were not previously seen with Ascl1 alone (FIG. 2, A to F).

IPA Induces Neurons with an RGC-Like Transcriptome

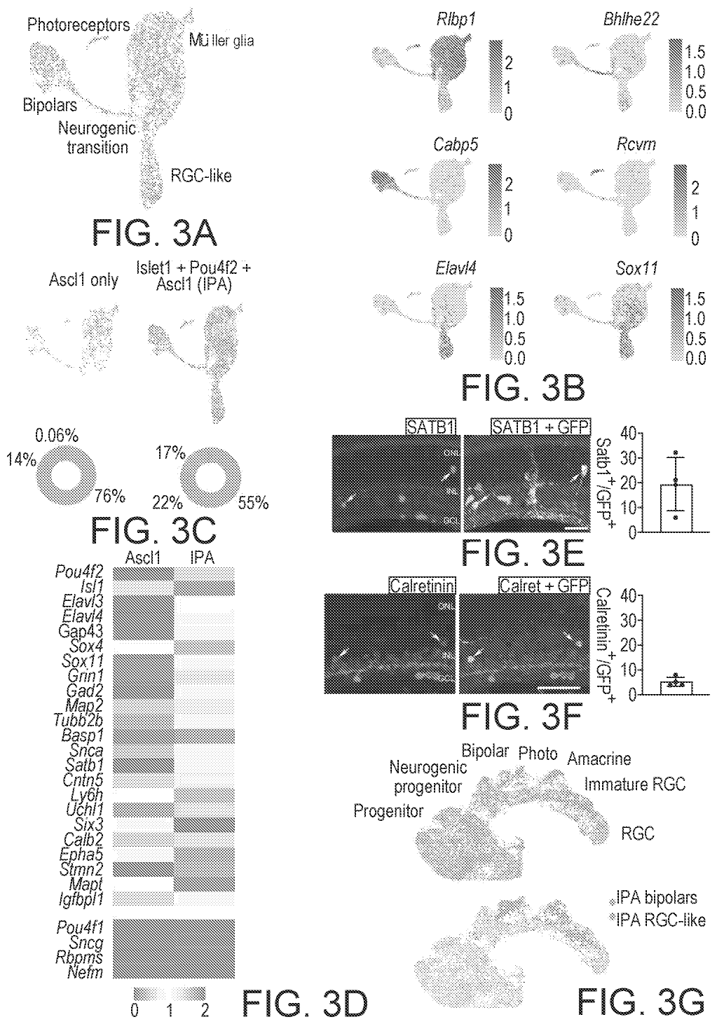

We next used scRNA-seq to analyze how Islet1 and Pou4f2 alter the phenotype of Ascl1-mediated MG reprogramming. Three weeks after initiating the IPA regeneration protocol, MG cells and their progeny were fluorescence-activated cell sorting (FACS)-purified and processed for scRNA-seq as previously described (5, 12). To directly compare the changes in cell fates induced by IPA with those caused by expression of Ascl1 alone, we used Seurat to integrate data from IPA treatment with previously obtained Ascl1-only reprogramming libraries (4) and clustered the cells (FIG. 3A). The combined data from the IPA experiment and the prior Ascl1 dataset were projected onto a single uniform manifold approximation and projection (UMAP) plot and clusters of cell types were identified by known marker genes (FIG. 3B).

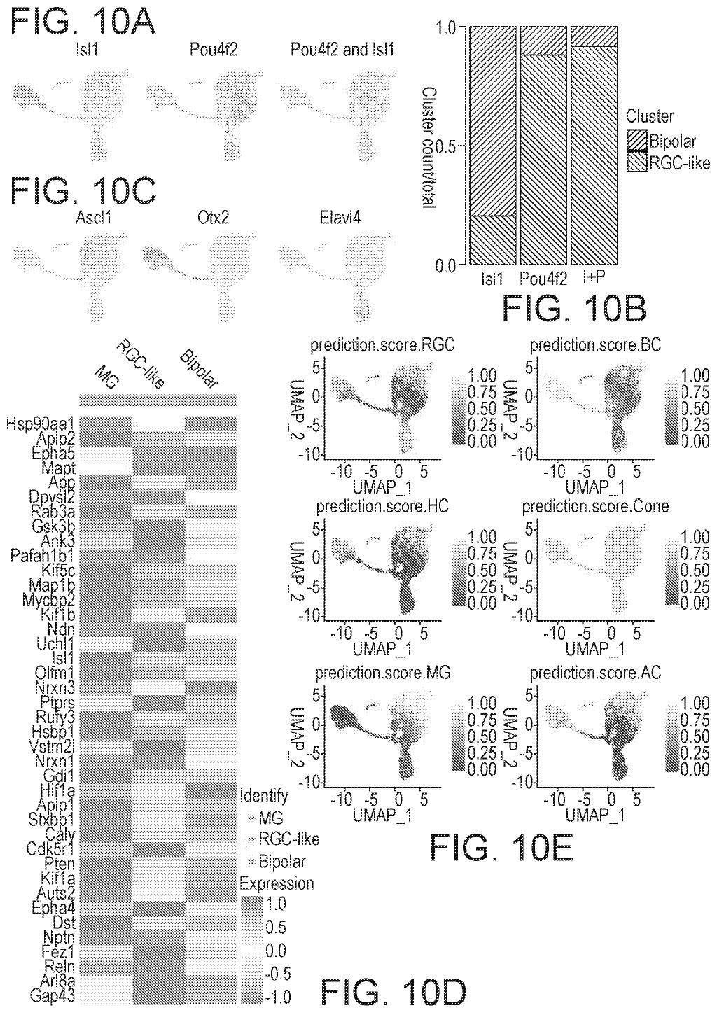

The combined UMAP plot of Ascl1-only versus IPA treatment contains clusters of cell types (e.g., MG, progenitors, and bipolar cells) that we have previously observed during Ascl1-mediated reprogramming (FIG. 3A). This analysis revealed two additional phenotypes unique to the IPA condition. First, the neurogenic efficiency of MG is increased over twofold with IPA versus Ascl1 only (FIG. 3C), which is consistent with our IHC data and suggests that the combination of these three TFs potently stimulates neurogenesis from adult MG. Second, a novel cluster appeared after IPA treatment that did not exist in the Ascl1-only condition (FIG. 3C). While cells expressing Islet1 but not Pou4f2 were found mostly in the bipolar cluster, cells expressing Pou4f2, with or without detectable Islet1, were largely found in the novel RGC-like cluster (FIG. 10A to 10C).

The new cluster of cells induced by IPA shows a high expression of genes characteristic of RGCs, such as Elavl4 and Sox11 (FIG. 3B) (20). In addition, we found that these IPA neurons expressed many genes found in the gene ontology (GO) terms “axon guidance,” “axon outgrowth,” and “axogenesis” (FIG. 10D). Because Islet1 and Pou4f2 are upstream of an RGC fate-inducing regulatory network, we assayed whether this combination of factors was able to induce multiple RGC genes in this cluster of MG-derived neurons. We used the label transfer feature of Seurat to broadly compare the transcriptome of the IPA neurons to a reference dataset of all major retinal neuron classes (21, 22). The novel cluster was classified as an RGC cluster (average prediction score=0.74), suggesting an overall transcriptomic similarity to native RGCs (FIG. 10E). Compared to Ascl1-induced neurons, a substantial number of RGC-associated genes were expressed in IPA-induced neurons (FIG. 3D). These include genes such as Sox4 and Sox11, which are redundantly required for RGC fate acquisition (23, 24), and the axon growth-associated gene Gap43, which is highly expressed in developing RGCs (25). We found that both Satb1 and Cntn5 were expressed in subsets of IPA-induced neurons. Satb1 is highly expressed in the ON-OFF direction-selective subtype of RGCs where it controls Cntn5 expression (26). Using IHC, we were able to confirm that IPA-induced neurons expressed Satb1 protein (FIG. 3E). In addition, the RGC and amacrine marker Calretinin (Calb2) was also detected at the RNA and protein levels (FIG. 3, C and F).

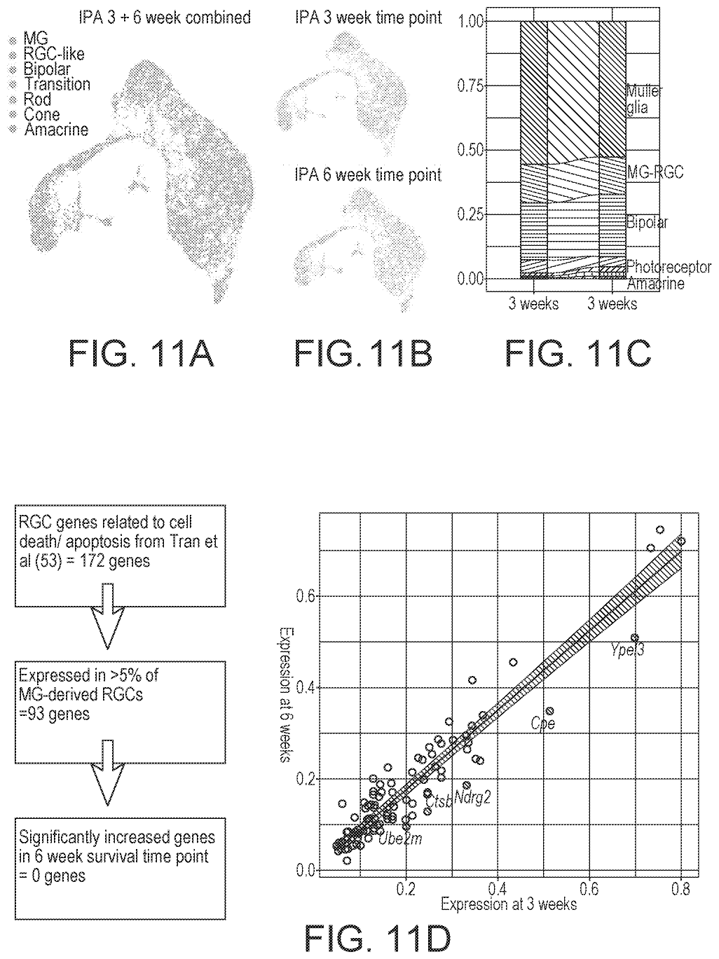

Despite this large suite of RGC genes expressed by the MG-derived RGC-like neurons, these cells fail to express some canonical RGC markers such as Pou4f1, Sncg, Rbpms, and Nefm (FIG. 3D). This suggests that the newborn neurons do not fully differentiate into mature RGCs. Consistent with this observation, when we integrated the scRNA-seq data of IPA-induced neurons with a dataset from E14 (embryonic day 14) embryonic mouse retina (21), we find that the MG-derived RGC-like neurons most closely resemble immature RGCs (FIG. 3G). We compared the IPA-induced neurons from a 3-week end point with a longer survival time point (6 weeks) and found that the regenerated RGC-like neurons were a stable population and did not show evidence of increased cell death or stress over this period (FIG. 11A-11D).

Simultaneous expression of Islet1-Pou4f2 with Ascl1 more uniformly induces an RGC state from MG in vitro

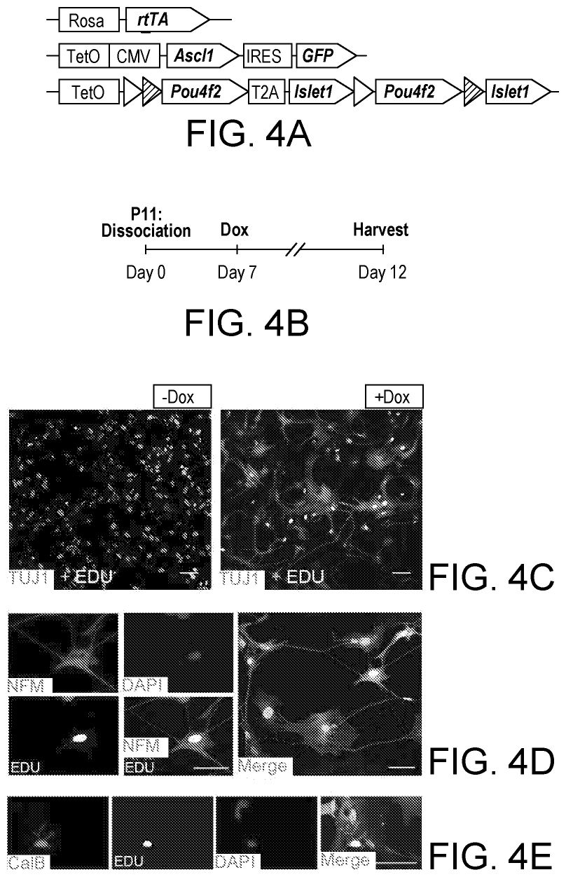

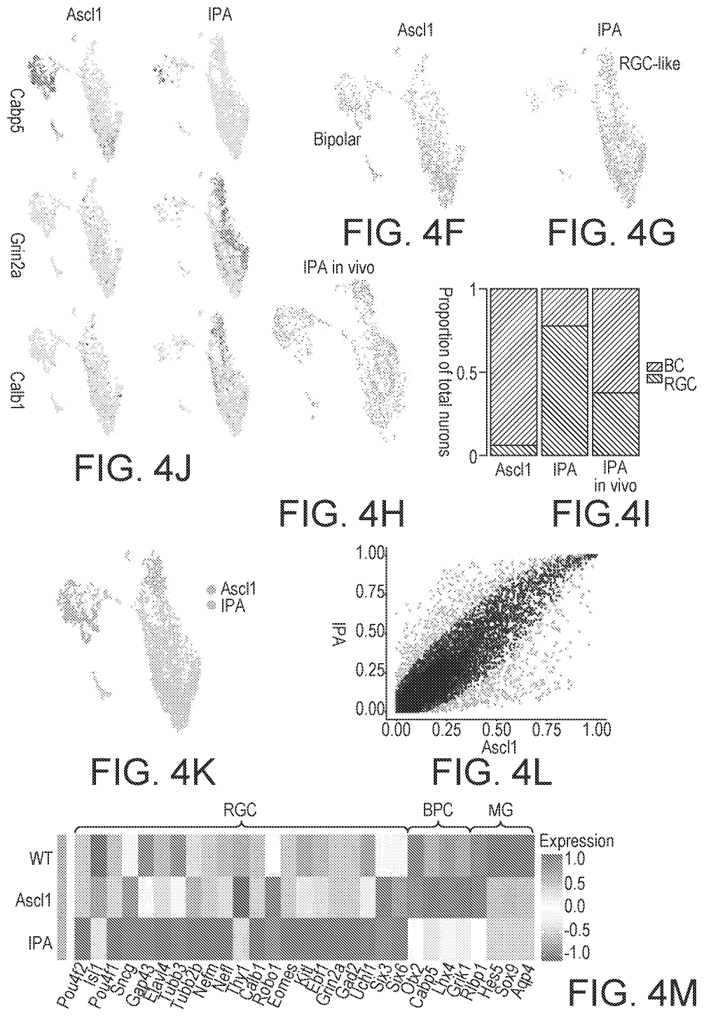

Because the tetO-Pou4f2 and Islet1 transgenic construct contains mutually exclusive loxP sites, most of the Cre-expressing MG in vivo expressed Ascl1 and either Pou4f2 or Islet1. While a small number of cells colabeled for both Pou4f2 and Islet1 in vivo (FIG. 1, C and D), to better assess the effect of overexpressing all three transgenes uniformly, we bred mice containing the tetO-Pou4f2-Islet1 and tetO-Ascl1-GFP cassettes to a germline Rosa26-rtTA line and then performed in vitro MG-reprogramming experiments (FIG. 4, A and B). MG were cultured from postnatal day 1111 mice for 7 days before passaging as previously described (10). The cultures obtained in this way are largely composed of MG, although some surviving neurons are observed (10). 5-Ethynyl-2′-deoxyuridine (EdU) was added to the medium to determine which cells are derived from proliferating MG and which cells were likely surviving neurons from the initial dissociation. After passage, doxycycline was added to the medium to induce transgene expression, and then cells were assayed with immunofluorescence and scRNA-seq (FIG. 4B).

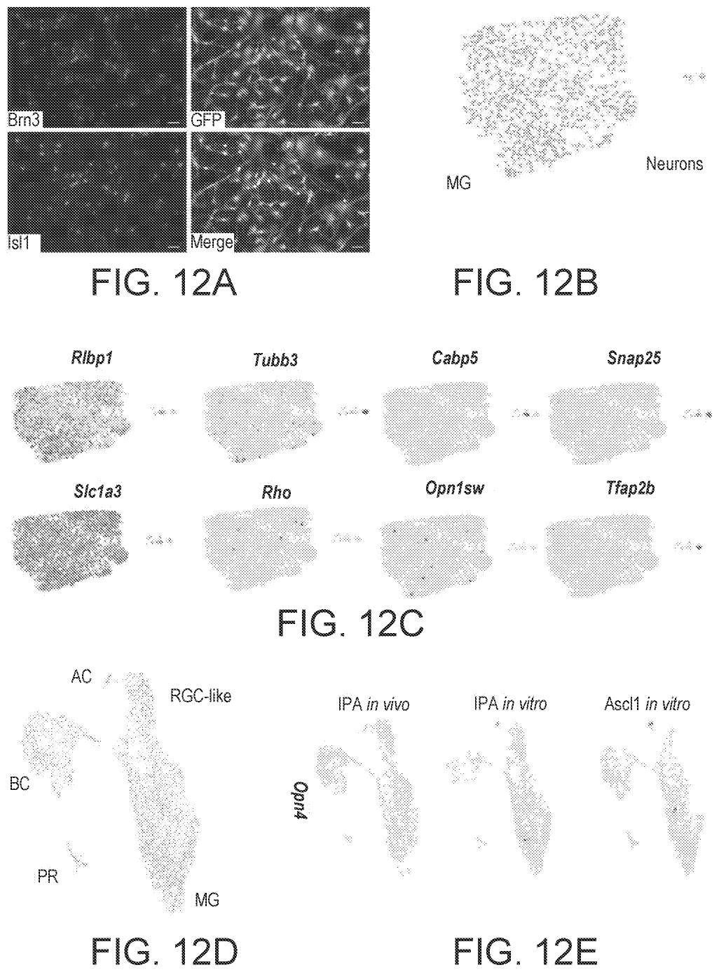

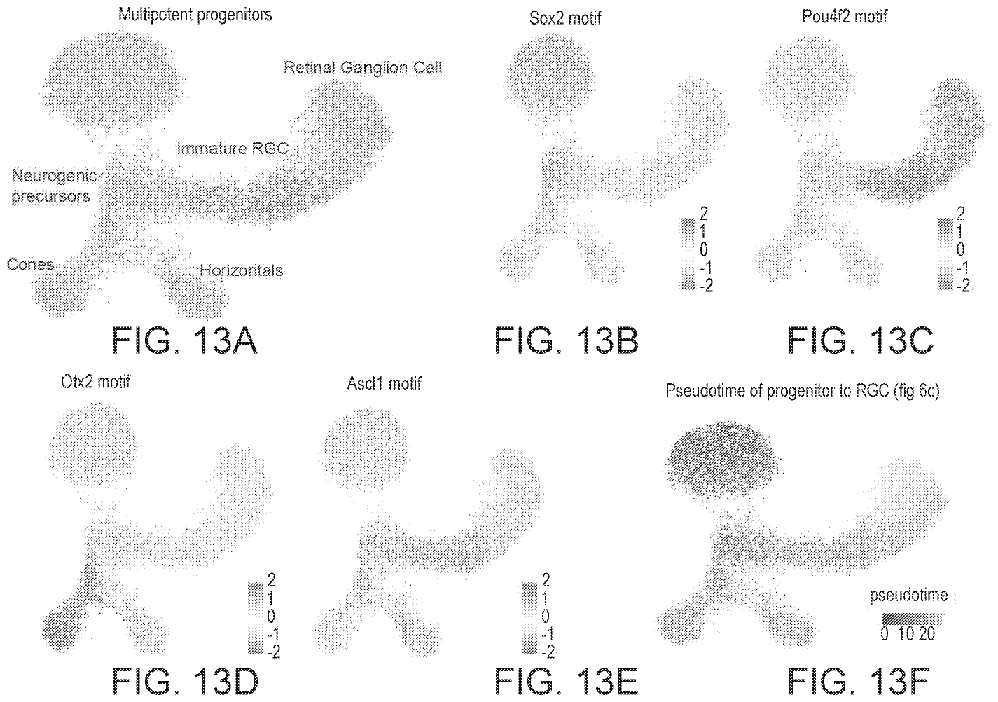

Analysis of the cultures after 5 days of treatment confirmed that the cells express the transgenes. Immunolabeling for the transgenes showed that cells coexpress Pou4f2, Islet1, and Ascl1 (FIG. 12A). The cells largely adopted a neuronal morphology, and immunolabeling demonstrated EdU+ cells that were colabeled with neuronal markers Tuj1 (FIG. 4C), Neurofilament (FIG. 4D), and Calbindin (FIG. 4E).

To determine how the overexpression of IPA differed from Ascl1 alone, we repeated the reprogramming experiment as described above, alongside sister MG cultures from tetO-Ascl1;Rosa26-rtTA mice, either with or without doxycycline treatment to activate transgene expression. We carried out scRNA-seq as described above for each sample. The untreated MG were largely homogeneous, with one glial cluster and a small cluster containing only a few surviving neurons (FIG. 12, B and C). To compare the cluster composition between the treatment conditions, as well as to the in vivo IPA dataset, we integrated all three together (FIG. 4, F to I, and FIG. 12D). We identified cells in both the Ascl1-only in vitro and IPA-reprogrammed MG in vitro that mapped to the neuron clusters from the IPA in vivo dataset. However, each treatment stimulated one main neuron cluster from MG; in the Ascl1 culture, the neurons most closely resembled bipolar cells (FIG. 4, F and 1), while in the IPA cultures, the neurons acquired an RGC-like fate (FIG. 4, G and 1), with a minority differentiating into bipolar cells. This differed from our observations in vivo, in which the bipolar cluster was similar in proportion to that of the Ascl1-only sample (FIGS. 1D and 4, F and H).

Next, we compared the gene expression profiles of the induced neurons in the IPA and Ascl1-only in vitro datasets. We observed that bipolar genes, such as Cabp5, were largely restricted to the Ascl1-only neuron cluster. RGC genes, such as Grin2a and Calb1, were found only in the IPA neurons (FIG. 4J). To identify unique marker genes expressed in the IPA neurons, we made a subset of all neuron populations in the IPA and Ascl1-only integrated dataset (FIG. 4K) and performed differential gene expression (DGE) analysis (FIG. 4L). We identified a number of RGC genes enriched in the IPA neurons, while bipolar genes were enriched in the Ascl1 neurons. In addition, we found some canonical markers of the RGC lineage that were not induced in vivo, such as Sncg, Nefm, and Pou4f1 that were induced in vitro (FIG. 4M). We also failed to detect cells expressing the intrinsic photosensitive RGC marker Opn4 (FIG. 12E). These results collectively suggest that concurrent overexpression of all three IPA factors biases MG-derived neurons to an RGC-like fate and induces more uniform RGC gene expression.

IPA Induces Neurons with Diverse Electrical Properties

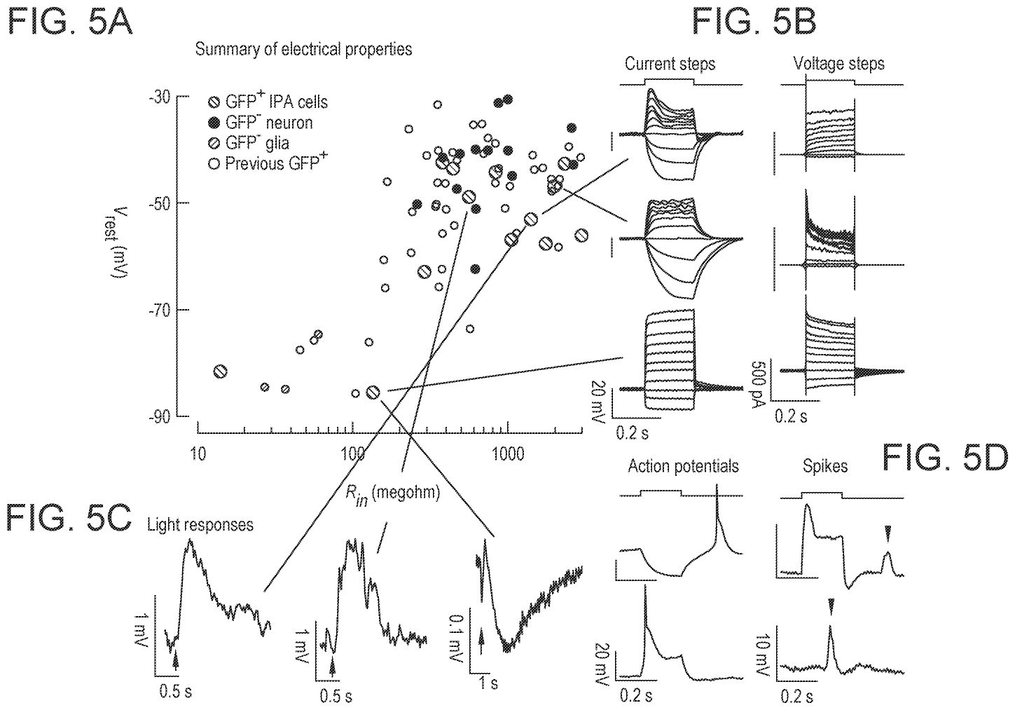

We found previously that Ascl1 can stimulate MG-derived neurons that have physiological characteristics of endogenous retinal neurons, particularly bipolar cells (4, 13). Because IPA treatment leads to a different molecular and morphological neuronal phenotype compared to Ascl1 alone, we characterized the light responses and electrical properties of these cells. We performed patch-clamp electrophysiology on GFP+ cells in retinal slices and whole mounts after our IPA in vivo regeneration paradigm. We measured responses to current and voltage steps and responses to light stimuli. We plotted the membrane resistance and resting potential of IPA-induced neurons compared to GFP-MG, endogenous neurons, and neurons from our previous regeneration strategies using Ascl1 or Ascl1:Atoh1 (FIG. 5A) (4, 5, 13). Glial cells have low membrane resistance, hyperpolarized resting potentials, and little in the way of voltage-activated conductance. Neurons have higher resistance, have less-negative resting potential, and express voltage-activated conductance.

Most of the IPA-induced neurons displayed resting potential and membrane resistance profiles similar to endogenous neurons; however, some of these cells still had glial-like hyperpolarized membrane potentials (FIG. 5A). This is consistent with our IHC and scRNA-seq analysis where a portion of glia does not reprogram after IPA treatment (see FIGS. 1 and 3). FIG. 5B shows examples of responses to families of current or voltage steps recorded from IPA-treated GFP+ cells. These cells exhibit a range of characteristics. Some have a neuronal phenotype and appear to express voltage-activated K+ conductance that limit the extent of depolarization to current steps, while others retain features of glia (FIG. 5B). Six of 16 recorded cells responded to brief light flashes, indicating that they established synaptic connections with other components of the retinal circuitry (FIG. 5C). Some cells responded to current steps by generating action potentials and others generated spiking activity likely representing Ca2+ spikes (FIG. 5D). This diversity of the physiological properties is consistent with the phenotypes observed in FIGS. 1 and 3. Notably, we did not observe action potentials, a distinct feature of RGCs, in our previous strategies to stimulate MG-derived neurons (4, 5, 13). Thus, IPA increases the diversity of the electrical properties of the MG-derived neurons, including generating some cells that can produce Na+ and/or Ca2+ action potentials.

### IPA Expression Remodels MG Chromatin to an Imperfect RGC-Like Fate

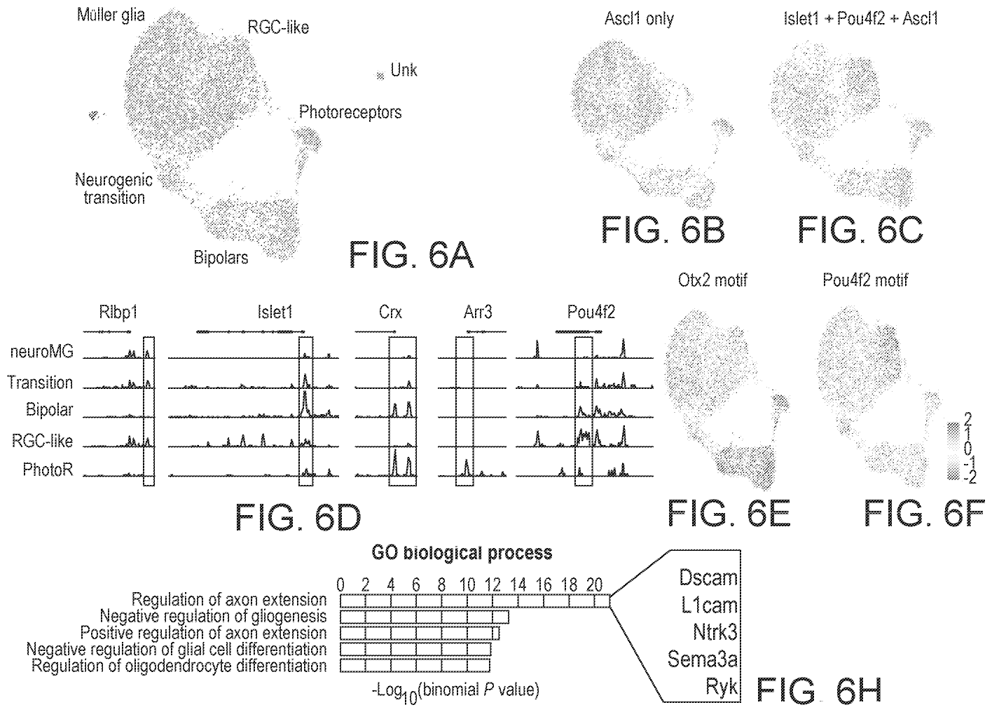

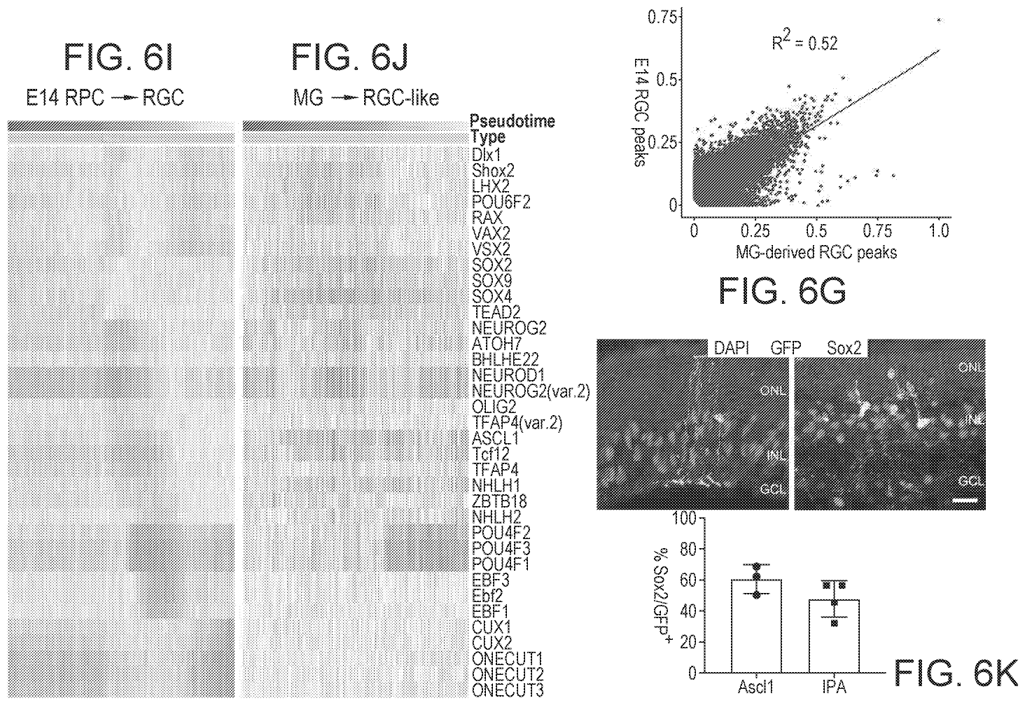

We next carried out single-cell assay for transposase-accessible chromatin sequencing (scATAC-seq) on Ascl1 versus IPA-reprogrammed MG to gain a better understanding of the mechanistic differences between these two reprogramming strategies. Mice were treated with the same in vivo retinal regeneration paradigm described in FIG. 3 but were processed for scATAC-seq instead of scRNA-seq. Nuclei (1692) from Ascl1 only and 2451 nuclei from IPA treatment passed our quality control metrics (see Materials and Methods). Single cells from these two treatments were then integrated and plotted as a UMAP to identify cell types (FIG. 6, A to C). Cell type clusters were identified by the pattern of accessible chromatin near genes identified with specific retinal cell types. Coverage plots show representative peaks for the groups we identified: MG (Rlbp1+), neurogenic transition (Islet1+), MG-derived bipolar cells (Crx+), induced RGC-like cells (Pou4f2+), and photoreceptors (Arr3+) (FIG. 6D). When the UMAP is split between the two treatment groups, it is clear that, while a large cluster of cells retain a MG phenotype, both the Ascl1 and the IPA conditions lead to MG-derived bipolar neurons (FIG. 6, B and C). However, the IPA treatment induces a unique cluster of cells that have accessible chromatin that most closely resemble RGCs (FIG. 6C). Consistent with accessibility patterns, the motif for Otx2 is enriched in the bipolar cluster, while the Pou4f2 motif is highly represented in the accessible chromatin of the IPA-unique cluster (FIG. 6F). This is consistent with our scRNA-seq findings and demonstrates that these MG-derived neurons have patterns of cis-regulatory regions consistent with their transcriptomic identity.

To determine how closely the chromatin of MG-derived RGCs resembles that of normal, developing RGCs, we generated a scATAC library from the E14 developing mouse retina to compare with the MG-derived RGCs (FIG. 13A-13F). The scatterplot in FIG. 6G shows that there is significant correlation between the accessible peaks in E14 RGCs and the MG-derived neurons; however, there are clearly many regions that are differentially accessible between cell types (FIG. 6G). When we assayed for GO enrichment to understand the types of genes that are more highly represented in nearby accessible chromatin in E14 RGCs than in MG-derived RGCs, many of the top terms were related to axon growth (FIG. 6H and table 2). This is consistent with the fact that many MG-derived RGCs do not extend long axons.

The current model of retinal development suggests that a cascade of TFs is sequentially activated when cells transition through multipotent progenitors, to neurogenic precursors, to their ultimate neuronal cell fate (27). Therefore, we sought to examine whether neurogenesis induced in MG recapitulates developmental cascades at the chromatin level. We used pseudotime to order cells in a projected lineage from the progenitor cells to RGCs in the E14 retina and compared this with a similar analysis of MG-derived RGCs and then analyzed the motif enrichment in accessible chromatin over pseudotime in a “cascade plot” (FIG. 6, I and J) (28). This analysis reveals key differences in the process of RGC development versus RGC production from MG. Normal development of RGCs shows down-regulation of progenitor TF motifs (Rax and Sox2) in accessible regions followed by a transient increase in regions with bHLH motifs (Atoh7), and then finally induction of regions with mature RGC TF motifs (Pou4f1/2/3, Ebf1, and Onecut). This has recently been described in both mouse and human (28, 29). By contrast, the sequential changes in motif representation in accessible chromatin in MG reprogrammed to generate neurons with IPA show clear differences from normal development. Although there is a reduction in progenitor gene motifs as cells acquire an RGC-like identity, the progenitor TFs are apparently never fully down-regulated, because their motifs persist in the accessible regions of MG-derived RGCs (FIG. 6J). In addition, although MG-derived RGCs show an increase in accessible bHLH motifs and a robust increase in Pou4f1/2/3 motif representation in accessible chromatin, the TFs that are presumably downstream of Pou4f1/3, such as Onecut and Ebf, are not sequentially activated (FIG. 6J). This epigenomic analysis suggests that RGC-like generation is imperfect from IPA-treated MG, in part because of the maintenance of accessibility at glial and progenitor regulatory regions. Consistent with this notion, the progenitor/MG marker Sox2 is still detectable by immunofluorescence 3 weeks after IPA reprogramming in GFP+ cells with neuronal morphology (FIG. 6K).

Atoh1 can Improve the Ability of IPA to Induce RGC-Like Cells from MG

The scRNA and scATAC-seq analysis revealed that, although IPA-induced neurons resembled RGCs, these cells lack some features of mature RGCs. One hypothesis for why this is the case is the persistence of glial and progenitor genes and chromatin accessibility (i.e., Sox, Rax, Vsx2, etc.) in the MG-derived neurons. Recently, we have reported that the Atoh class of TFs, when combined with Ascl1, can potently stimulate neurogenesis (13); the combination of Ascl1:Atoh1 results in nearly 80% of transgene-expressing MG acquiring a neural identity. Therefore, we tested whether combining Atoh1 with IPA reprogramming could improve the regeneration of RGCs from MG in vivo.

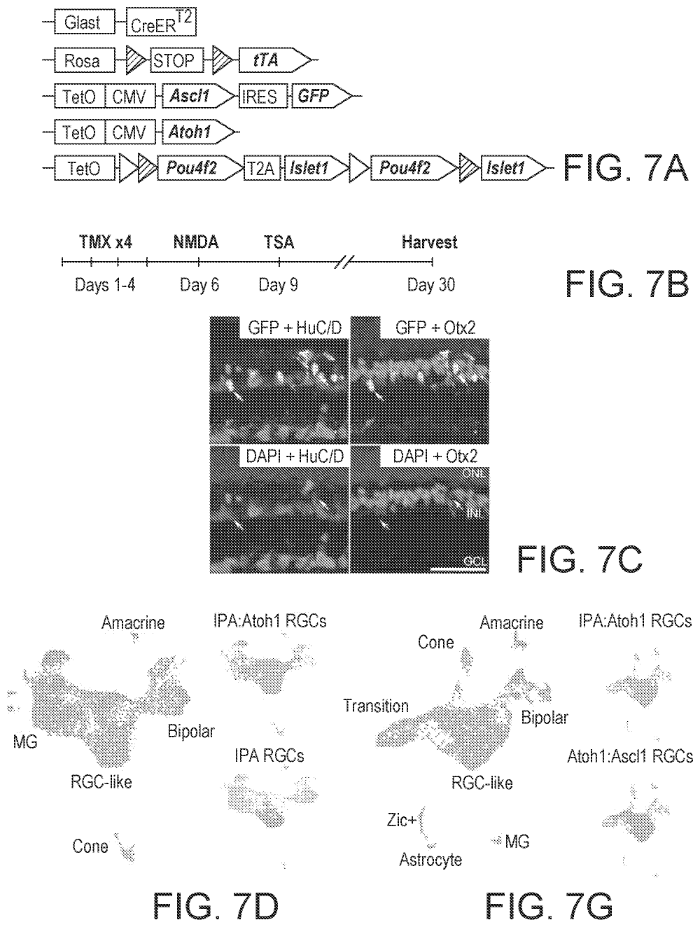

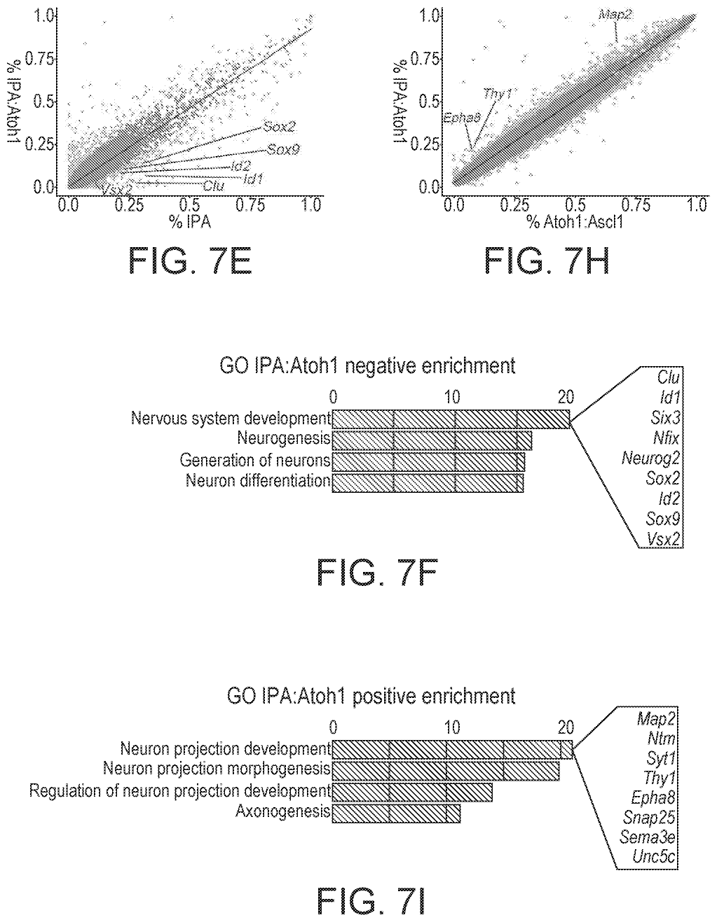

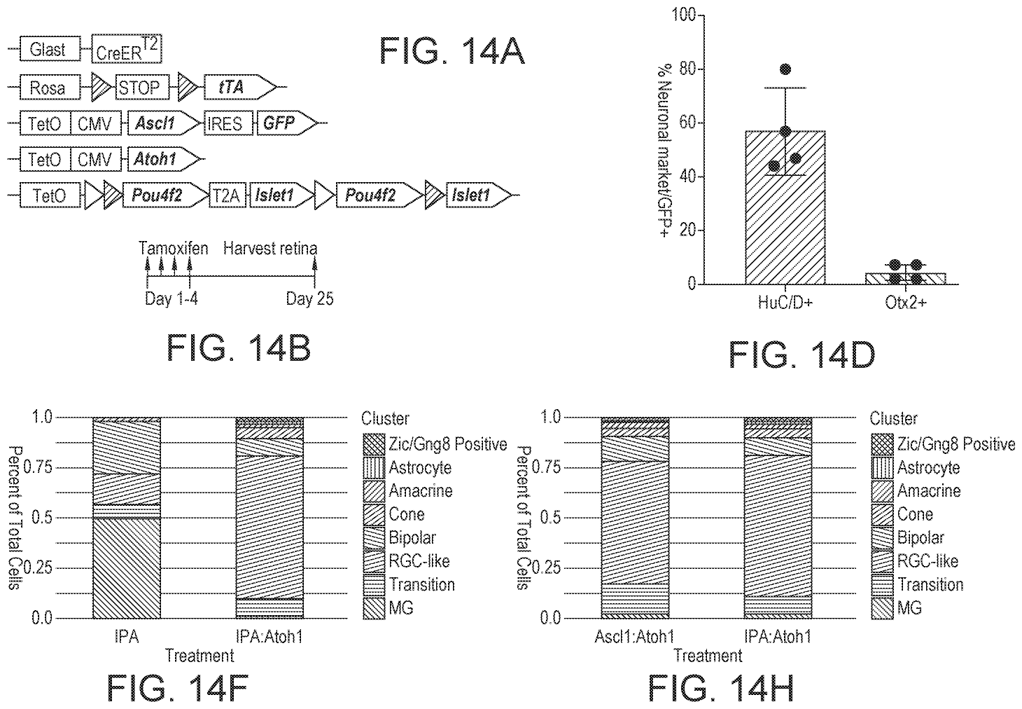

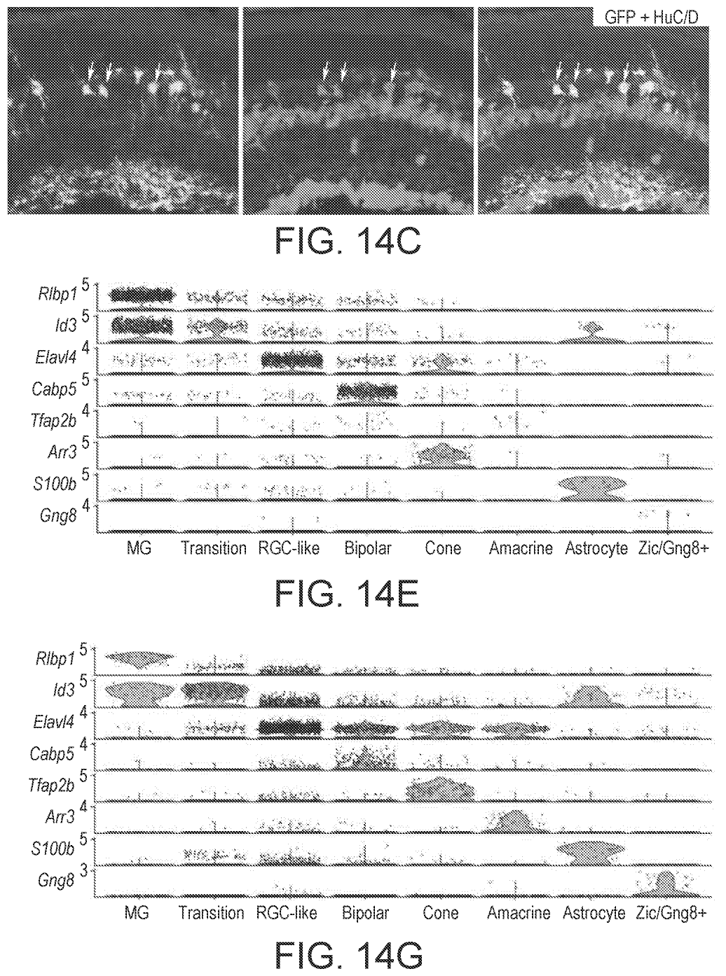

We crossed mice containing a tetracycline-inducible Atoh1 to the IPA strain and carried out the regeneration paradigm as described in FIG. 1; we then performed scRNA-seq and IHC analysis of MG progeny as described above (FIG. 7, A and B). Consistent with our previous findings using Atoh1, the expression of IPA and Atoh1 in MG caused most of the MG progeny to acquire a neuronal identity (13). IHC revealed that most GFP+MG-derived neurons expressed the ganglion/amacrine marker HuC/D and lacked expression of the bipolar marker Otx2 (FIG. 7C). Adding Atoh1 to the combination of IPA factors also bypasses the requirement for retinal injury to induce MG neurogenesis (FIG. 14A-14D). We next performed scRNA-seq on regenerated cells treated from the IPA:Atoh1 condition and integrated them with cells from the IPA-only treatment group to determine whether the addition of Atoh1 may improve RGC generation after retinal damage (FIG. 7D). This analysis revealed that MG-derived neurons from the IPA:Atoh1 mice were most similar to the MG-derived RGCs from the IPA condition, while a smaller proportion of bipolar, cone, and amacrine cells were also present (FIG. 7D and FIGS. 14E and 14F).

To assess whether Atoh1 overexpression reduces the progenitor signature of MG-derived neurons after IPA expression, we formed a subset of the RGC-like cells from each dataset for comparison. DGE analysis showed that, in the IPA:Atoh condition, the RGC-like cells showed a greater decrease in expression of progenitor and glial genes than similar cells from the IPA only condition (FIG. 7E). This was further confirmed with GO analysis, where IPA:Atoh1 reduced genes associated with neural progenitors compared to the IPA-only condition (FIG. 7F), consistent with the hypothesis that Atoh1 promotes maturation of the RGC-like cells.

We have previously shown Ascl1:Atoh1 generates immature RGC-like cells from MG (13). We hypothesized that the addition of IPA factors to Atoh1 might induce markers of more mature RGCs. We compared IPA:Atoh1 with previously generated Ascl1:Atoh1 datasets and found a similar composition of cell types (FIG. 7G and FIG. 14E to 14H). However, we found that the addition of IPA to Atoh1 increased RGC-like cells by 15% and resulted in a corresponding decrease in bipolar and progenitor-like cells (FIGS. 14G and 14H). DGE analysis revealed that the addition of IPA to Ascl1:Atoh1 leads to an enrichment of RGC genes such as Map2 and Thy1 (FIG. 7H). Furthermore, we found that the top GO terms enriched in the IPA:Atoh1 were related to neuron projection development and axonogenesis (FIG. 7I), with many genes specifically related to axon guidance (e.g., Epha8 and Sema3e) and synaptogenesis (e.g., Syt1 and Snap25).

TABLE 2
Genes associated with peaks enriched in
E14 RGCs vs. MG-derived RGC-like cells

| negative | | | |
|---|---|---|---|
| regulation of | negative | positive | regulation of |
| glial cell | regulation of | regulation of | oligodendrocyte |
| differentiation | gliogenesis | axon extension | differentiation |
| Ctnnb1 | Abcc8 | Adnp | Ctnnb1 |
| Dab1 | Adcyap1 | Cdh4 | Cxcr4 |
| Dlx1 | Ascl2 | Disc1 | Dicer1 |
| Dlx2 | Ctnnb1 | Dscam | Dlx1 |
| Drd3 | Dab1 | Fn1 | Dlx2 |
| Dusp10 | Dicer1 | Islr2 | Drd3 |
| Hes1 | Dlx1 | L1cam | Dusp10 |
| Hes5 | Dlx2 | Limk1 | Dusp15 |
| Hmga2 | Drd3 | Map1b | Hes1 |
| Id2 | Dusp10 | Mapt | Hes5 |
| Id4 | Hes1 | Ndel1 | Id2 |
| Kdm4a | Hes5 | Nrg1 | Id4 |
| Lin28a | Hmga2 | Nrp1 | Lingo1 |
| Lingo1 | Id2 | Ntn1 | Olig2 |
| Mycn | Id4 | Ntrk3 | Rheb |
| Nkx6-1 | Idh2 | Pafah1b1 | Shh |
| Nkx6-2 | Kdm4a | Pou4f2 | Tcf712 |
| Nog | Lin28a | Rab11a | Tenm4 |
| Ntrk3 | Lingo1 | Rufy3 | Tnfrsf21 |
| | Mycn | Sema5a | Zfp365 |
| | Nkx6-1 | Sema7a | Zfp488 |
| | Nog | Srf | |
| | Ntrk3 | Vegfa | |
| | Pitx3 | | |
| | Ptn | | |
| | Ski | | |
| | Sox10 | | |
| | Sox11 | | |

regulation of axon extension

| | | | |
|---|---|---|---|
| Abl1 | Islr2 | Olfm1 | Sema3a |
| Adnp | L1cam | Pafah1b1 | Sema3f |
| Barhl2 | Limk1 | Plxna1 | Sema3g |
| Cdh4 | Map1b | Plxna2 | Sema4f |
| Cdk5 | Mapt | Plxna4 | Sema5a |
| Cdk5r1 | Mgll | Pou4f2 | Sema7a |
| Clasp2 | Ndel1 | Ptprs | Srf |
| Cttn | Nkx6-1 | Rab11a | Tnr |
| Disc1 | Nrg1 | Rtn4 | Vegfa |
| Draxin | Nrp1 | Rtn4r | Wdr36 |
| Dscam | Ntn1 | Rufy3 | Wnt3 |
| Fn1 | Ntrk3 | Ryk | Wnt5a |

### DISCUSSION

Nonmammalian adult vertebrates can regenerate neurons in many regions of their central nervous system (CNS). For example, after tail amputation in larval frogs and some adult urodeles, the radial glial cells of the spinal cord acquire a pattern of gene expression similar to neuronal precursors and go on to proliferate and regenerate an apparently normal spinal cord (30). Similarly, in the retina and brain of zebrafish, glia respond to injury by activating a progenitor-like gene expression program of TFs (31). These glia-derived progenitor cells undergo multiple rounds of mitotic cell divisions, and the progeny differentiate into the range of neuron types that can restore function in the brain and retina (32).

Reexpressing developmentally active TFs in adult mammalian glia can trigger a regenerative process in these cells that, in many ways, resembles what is found in fish and amphibians. For example, after retinal injury, the transgenic overexpression of the proneural TF Ascl1, combined with histone deacetylase inhibition, can stimulate MG to acquire a progenitor-like state with the capacity of generating bipolar neurons (4). The MG-derived neurons differentiate to the point that they make synapses with the surrounding neuronal circuitry and respond to light. In addition to lineage tracing the neurons to validate their glial derivation, we have used EdU labeling to show their adult origin and have profiled the cells using scRNA-seq, and scATAC-seq, to observe intermediate states between glial progenitor and regenerated neurons (5, 12, 13). Together, this validates that TFs can reprogram glia in the adult CNS to generate neurons.

Although Ascl1 induces MG to adopt many features of retinal progenitors, including proliferative neurogenesis and a transcriptional and epigenetic landscape similar to developmental progenitors, not all developmentally appropriate Ascl1 targets are induced in MG-derived progenitor cells, and the neuronal output from Ascl1 MG is restricted to primarily bipolar neurons (5). Thus, we reasoned that additional TFs might be required to properly steer MG-derived progenitors to specific types of neurons. This is particularly important for endogenous regeneration strategies because most blinding diseases are the result of deficits in a particular neuronal subtype. For example, glaucoma is primarily caused by the death of RGCs.

RGCs are generated during development by a cascade of TFs, characterized by the initial expression of Atoh7 and the downstream expression of additional TFs, such as Pou4f1/2 and Islet1 (33). Atoh7 is necessary for proper RGC fate by inducing these downstream stabilizing TFs (34-36). Two of these downstream TFs, Pou4f2 and Islet1, are required for proper RGC fate specification (18, 37, 38), and ectopic expression of Pou4f2 and Islet1 in the Atoh7 null retina is sufficient to rescue the RGC fate (19). By taking advantage of the wealth of knowledge of normal transcriptional regulation of the RGC fate, we were able to test whether members of this TF cascade can reinitiate the genesis of these cells from the Ascl1-induced MG-derived progenitors.

We report here that the RGC fate-inducing factors, Islet1 and Pou4f2, along with Ascl1, can induce MG neurogenesis toward an RGC-like fate. Using IHC, we show that the MG-derived neurons, from IPA-expressing MG, express protein markers and morphological features of RGCs. At the physiological level, these IPA-induced, MG-derived neurons display neuronal resting membrane potentials and a subset generated voltage-gated action potentials, consistent with an RGC-like fate. Last, our molecular analysis using scRNA-seq and scATAC-seq show that, at the transcriptomic and epigenetic levels, IPA-induced MG-derived neurons most closely resemble immature RGC cells.

Although the expression of the IPA transgenes in MG collectively reprograms a subset of the cells to an RGC-like fate, this combination does not activate the full complement of TFs required for mature RGCs. We find both in the scRNA-seq and in the scATAC-seq datasets that some mature RGC genes fail to be induced; instead, the MG-derived neurons retain expression of some progenitor/glial genes and a resulting chromatin landscape intermediate between progenitors and RGCs. The progenitor state in MG can be repressed by the expression of Atoh1 in addition to IPA, consistent with the role for atonal TFs in promoting neuronal differentiation, and this allows further differentiation of the MG-derived RGCs. Together, our results show that the ectopic expression of developmental TFs that are integral to a cell type-specific trajectory can reprogram MG toward that same trajectory. This suggests an overall approach where the complementary and sequential actions of TFs in development of specific neuronal types can be used to regenerate more mature neurons. It remains to be seen whether expression of TFs in their normal developmental sequence will provide a more effective strategy for reprogramming.

It is interesting that the RGC-like neurons derived from MG are most frequently found in the inner nuclear layer (INL), instead of the ganglion cell layer (GCL). Although a small percentage of RGCs is normally found in the INL in mice, most migrate to the GCL. It is possible that the inner plexiform layer provides a barrier to migration of the RGC-like cells, or alternatively, they may lack the cues for appropriate migration. Although the scRNA-seq data show MG-derived RGC-like cells express many RGC genes, they may lack some critical migration program. Nevertheless, some of these cells connect with the existing neural circuitry and respond to light, and it may be that appropriate connectivity can be established without proper somal location.

Although we also did not observe robust axonal outgrowth directed to the optic nerve, we find that many genes important for axon growth and guidance are expressed in the RGC-like cells. It is possible that some key guidance factors are not expressed in these cells or, alternatively, that the adult retinal environment no longer expresses the guidance factors needed to direct axons to the optic nerve head. RGC transplant experiments have found that, in some cases, the RGC axons project ectopically, suggesting that the microenvironment of the adult retina may not fully reflect that of the developing retina; future studies will be needed to better understand the interplay between cell-autonomous factors and nonautonomous factors in regeneration.

Over the past decade, a number of reports have suggested that other developmental TFs such as Sox2, Pax6, NeuroD1, Neurog2, and Ascl1 can stimulate neurogenesis from glia both in vitro and in the brain (39). However, the interpretation of many of these reports has been clouded by the finding that lineage tracing new neurons is unreliable using existing Adeno-Associated Virus (AAV) paradigms and often leads to endogenous neurons being misidentified as glial derived (40, 41). Nevertheless, some of the earlier studies that used alternative methods to trace glial-derived neurons were able to show that reprogramming of astrocytes to neurons is possible, and proneural TFs, including Ascl1, are among the neurogenic stimulants (42-45). Thus, it is possible that the strategy of combining TFs from a specific neuronal lineage, as we have used for the retina, will have applicability throughout the CNS.

Overall, this study provides a proof of concept that TFs used to specify cell fate in development can be harnessed to initiate similar fate acquisition from reprogrammed glia cells in the adult CNS. The re-engineering process of cell fate acquisition during endogenous regeneration strategies will be key in guiding neural replacement appropriate for specific neurodegenerative diseases.

### REFERENCES

1. L. Todd, T. A. Reh, Cold Spring Harb. Perspect. Biol. 14, a040816 (2022).
2. A. Hamon, et al. Dev. Dyn. 245, 727-738 (2016).
3. A. Bringmann, et al. Prog. Retin. Eye Res. 28, 423-451 (2009).
4. N. L. Jorstad, et al. Nature 548, 103-107 (2017).
5. N. L. Jorstad, et al. Cell Rep. 30, 2195-2208.e5 (2020).
6. T. Hoang, at al. Science 370, eabb8598 (2020).
7. E. M. Rueda, et al. Cell Rep. 27, 1637-1649.e6 (2019).
8. K. Yao, et al. Cell Rep. 17, 165-178 (2016).
9. K. Yao, et al. Nature 560, 484-488 (2018).
10. J. Pollak, et al. Development 140, 2619-2631 (2013).
11. Y. Ueki, et al. Proc. Natl. Acad. Sci. U.S.A. 112, 13717-13722 (2015).
12. L. Todd, et al. Cell Rep. 33, 108507 (2020).
13. L. Todd, et al. Cell Rep. 37, 109857 (2021).
14. C. Powell, et al. Sci. Rep. 6, 24851 (2016).
15. F. D. D'Orazi, et al. J. Comp. Neurol. 528, 2816-2830 (2020).
16. G. Beykin, et al. Prog. Retin. Eye Res. 80, 100875 (2021).
17. Y. Elshatory, et al. J. Comp. Neurol. 503, 182-197 (2007).
18. L. Gan, et al. Proc. Natl. Acad. Sci. U.S.A. 93, 3920-3925 (1996).
19. F. Wu, et al. Proc. Natl. Acad. Sci. U.S.A. 112, E1559-E1568 (2015).
20. A. Usui, et al. Development 140, 740-750 (2013).
21. B. S. Clark, et al. Neuron 102, 1111-1126.e5 (2019).
22. T. Stuart, et al. Cell 177, 1888-1902.e21 (2019).
23. K. C. Chang, et al. J. Neurosci. 37, 4967-4981 (2017).
24. Y. Jiang, et al. J. Biol. Chem. 288, 18429-18438 (2013).
25. T. A. Reh, et al. J. Neurobiol. 24, 949-958 (1993).
26. Y.-R. Peng, et al. Neuron 95, 869-883 e866 (2017).
27. E. A. Bassett, et al. Trends Neurosci. 35, 565-573 (2012).
28. C. Finkbeiner, et al. Cell Rep. 38, 110294 (2022).
29. P. Lyu, et al. Cell Rep. 37, 109994 (2021).
30. A. D. Kakebeen, et al. eLife 9, e52648 (2020).
31. B. W. Lindsey et al. Prog. Neurobiol. 170, 99-114 (2018).
32. D. Goldman, Nat. Rev. Neurosci. 15, 431-442 (2014).
33. F. Wu, et al. Nat. Commun. 12, 1465 (2021).
34. N. L. Brown, et al. Development 128, 2497-2508 (2001).
35. J. N. Kay, et al. Neuron 30, 725-736 (2001).
36. J. Brodie-Kommit, et al. Sci. Adv. 7, eabe4983 (2021).
37. X. Mu, et al. Proc. Natl. Acad. Sci. U.S.A. 105, 6942-6947 (2008).
38. L. Pan, et al. Development 135, 1981-1990 (2008).
39. R. Bocchi, et al. Neuron 110, 366-393 (2022).
40. S. Blackshaw, et al. J. Clin. Invest. 131, e146134 (2021).
41. L.-L. Wang, et al. Cell 184, 5465-5481.e16 (2021).
42. C. Lentini, et al. Cell Stem Cell 28, 2104-2121.e10 (2021).
43. O. Torper, et al. Proc. Natl. Acad. Sci. U.S.A. 110, 7038-7043 (2013).
44. C. Heinrich, et al. Stem Cell Rep. 3, 1000-1014 (2014).
45. N. Mattugini, et al. Neuron 103, 1086-1095.e5 (2019).
46. A. Butler, et al. Nat. Biotechnol. 36, 411-420 (2018).
47. A. T. Satpathy, et al. Nat. Biotechnol. 37, 925-936 (2019).
48. T. Stuart, et al. Nat. Methods 18, 1333-1341 (2021).
49. Y. Zhang, et al. Genome Biol. 9, R137 (2018).
50. S. Neph, et al. Bioinformatics 28, 1919-1920 (2012).
51. J. Cao, et al. Nature 566, 496-502 (2019).
52. C. Y. McLean, et al. Nat. Biotechnol. 28, 495-501 (2010).
53. N. M. Tran, et al. Neuron 104, 1039-1055.e12 (2019).

### Example 2: Promotion of RGC Production Using Transcription Factors Specific to Developing Retinal Cells

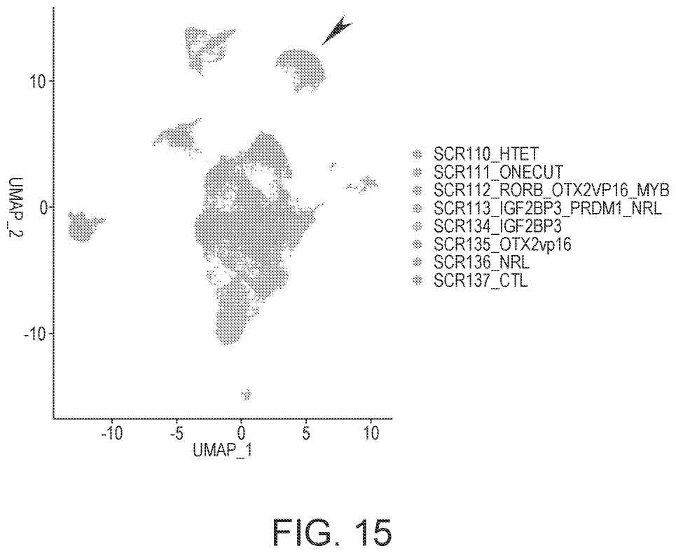

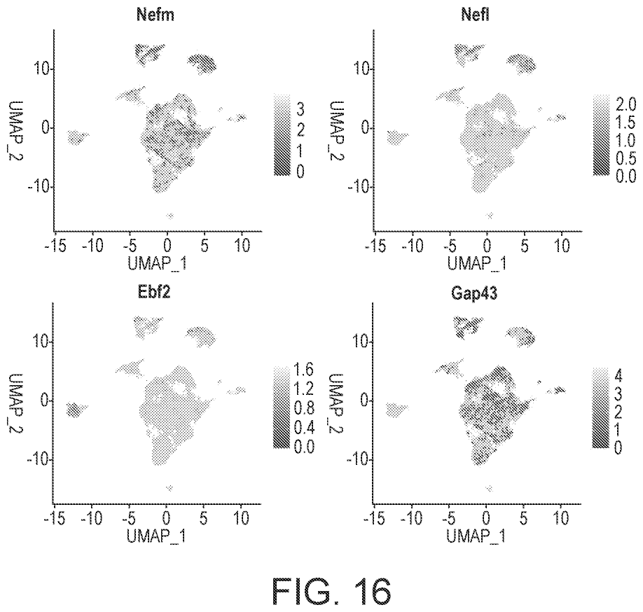

To determine which transcription factors could aid in promoting the production of retinal ganglion cells from Müller glia, we used lentiviruses to induce the expression of several transcription factors that are expressed in developing retinal cells, but not in Müller glia. The Müller glia were grown in cell culture and infected with the viruses. The cells were then cultured for 5-7 days and then processed for single cell RNAseq to determine their fates. We found that of all the factors tested, Onecut1 (arrow in FIG. 15) was able to induce the Müller glia to generate new cell with the characteristic gene expression of RGCs, including Nefm, Nefl, Ebf1 and Gap43 (FIG. 16).

### Example 3: AAV Delivered Reprogramming Transcription Factors can Induce Neurogenesis

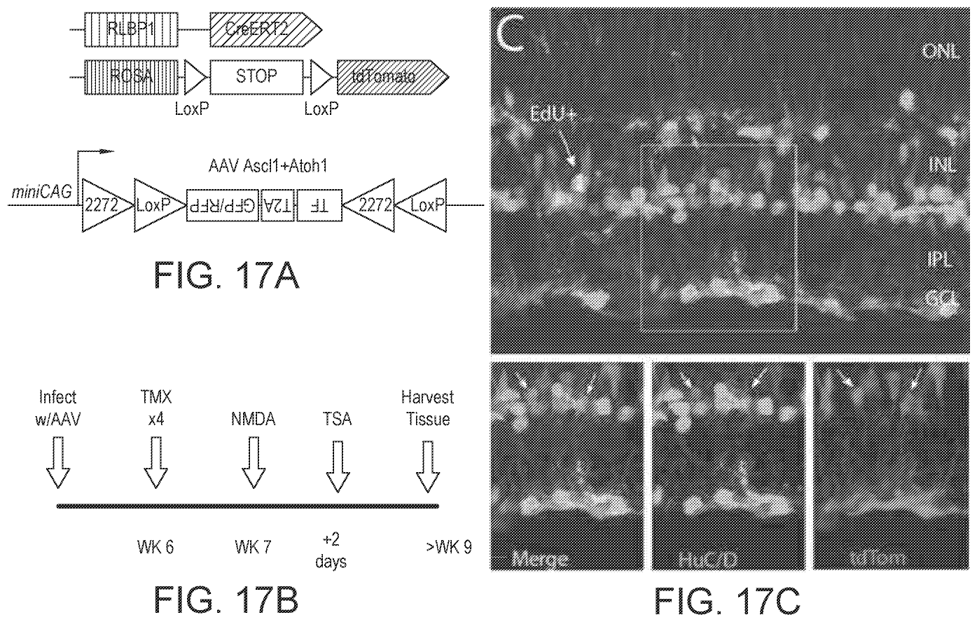

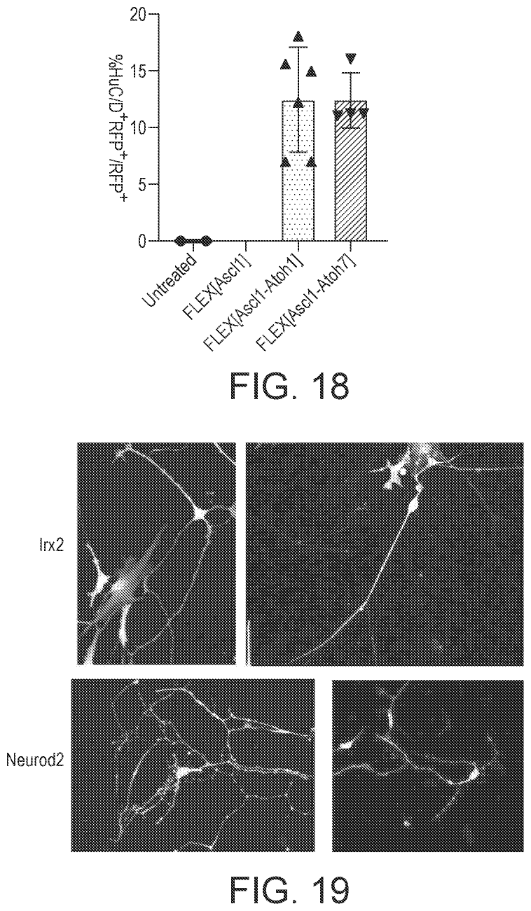

FIGS. 17A-17C. illustrate the protocol used to demonstrate that the lineage of HuC/D+ neurons was traced from MG by tdTomato. Immunofluorescent markers showed successful delivery of reprogramming transcription factors resulting in HuC/D+ neurons reprogrammed from MG cells. As shown in FIG. 18, both Atoh1 and Atoh7 delivered by AAV can induce HuC/D+ neurons. Atoh1 and Atoh7 are about the same in efficiency. AAV delivery of reprogramming factors under these conditions was much less efficient than transgenic expression.

We have tested 5 additional RGC transcription factors: Irx2, Irx5, Neurod2, Ebf1, Tcf3. Of these, Irx2 and Neurod2 promote axon growth in IPA reprogrammed MG (FIG. 19). This shows that certain RGC transcription factors can increase some specific RGC genes.

### Example 4: Human Muller Glia can be Generated In Vitro from Fetal Retina or Pluripotent Stem Cells

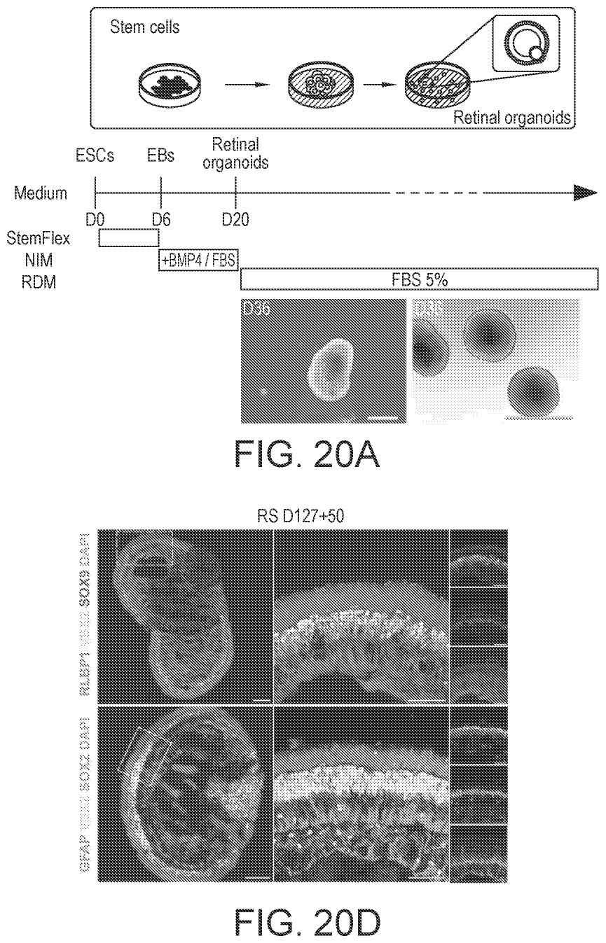

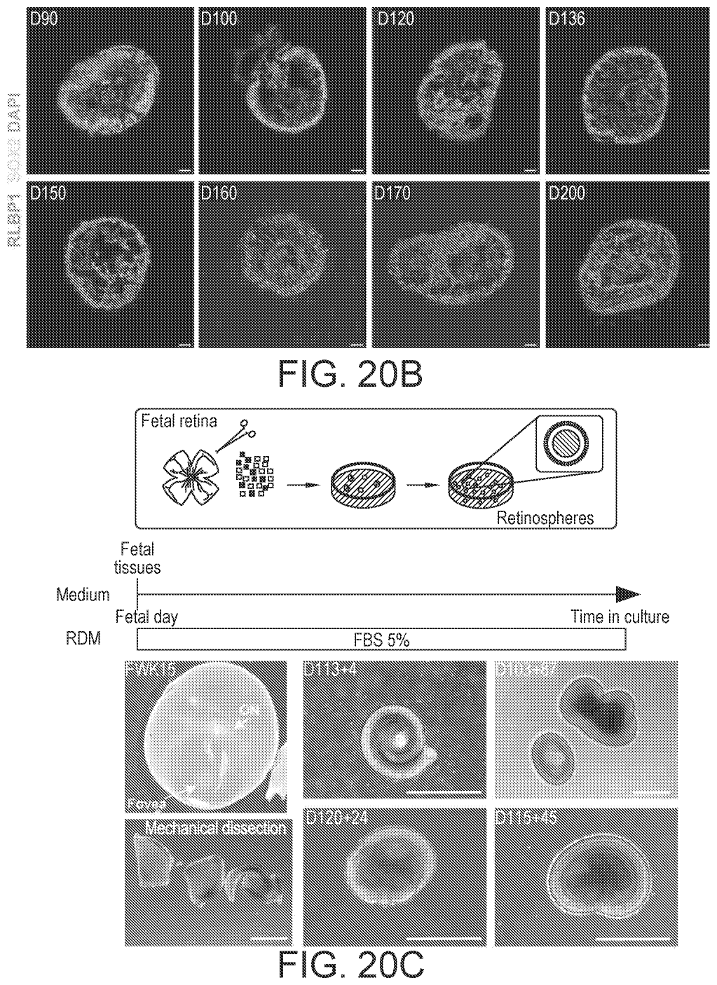

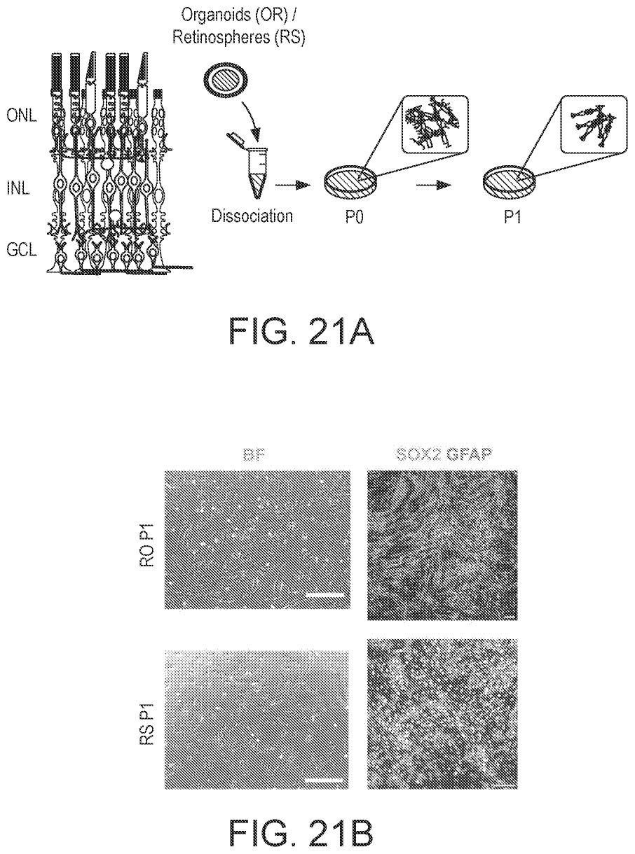

The protocol for Embryonic Stem cell (ESC) differentiation to generate retinal organoids (RO) is illustrated schematically in FIG. 20A. Images of MG development in retinal organoids over time labeled with RLBP1 and SOX2 are shown in FIG. 20B. The upper panel of FIG. 20C shows the schematic protocol for the generation of retinospheres (RS), and the lower panel shows images of retinospheres made from several fetal retinas and cultured for various times as labeled. Characterization of the MG in RS with RLBP1, VSX2, SOX9, SOX2 and GFAP is shown in FIG. 20D.

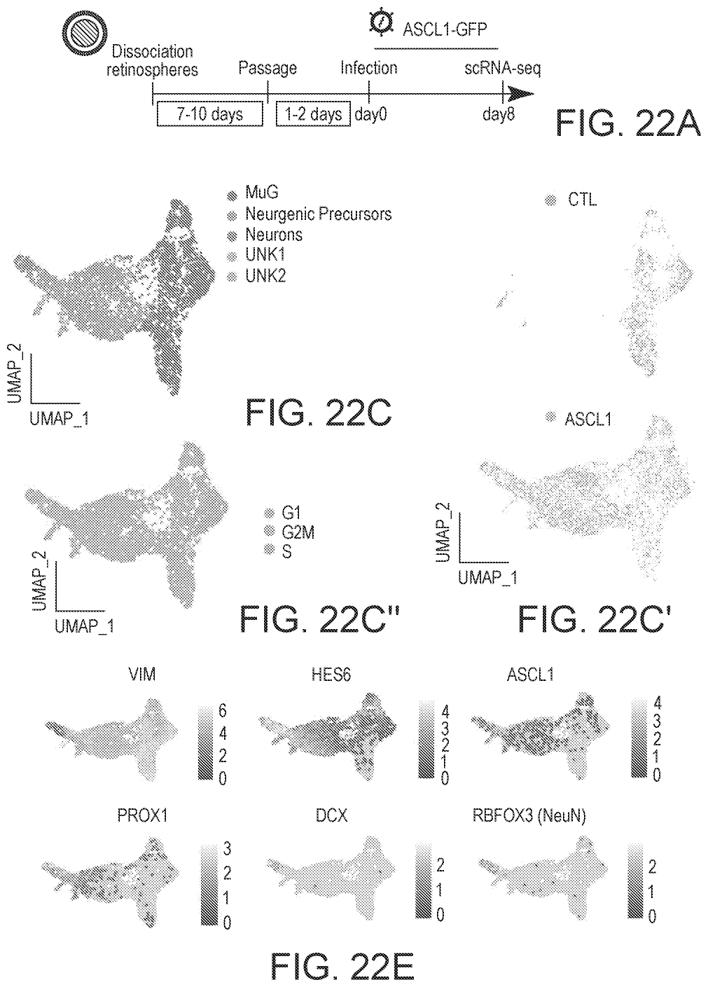

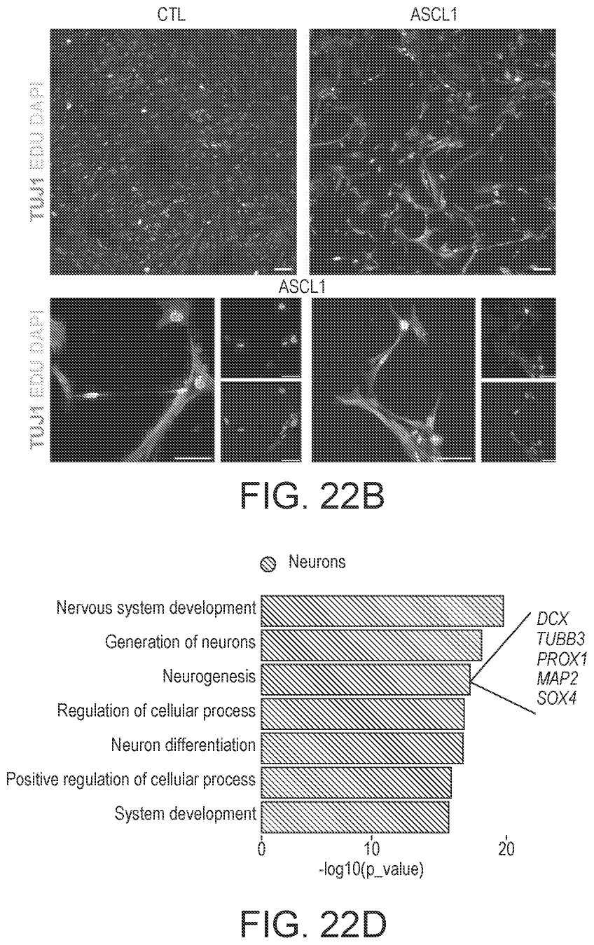

Human MG can be reprogrammed with ASCL1 to generate neurons in dissociated cultures (FIG. 22). These neurons express some pan neuronal markers such as DCX and TUJ1, but are not mature. Human MG can be derived from either retinal organoids or fetal human retina; both glial sources respond similarly to ASCL1.

### Example 5: Promoters for Driving Expression in Muller Glia

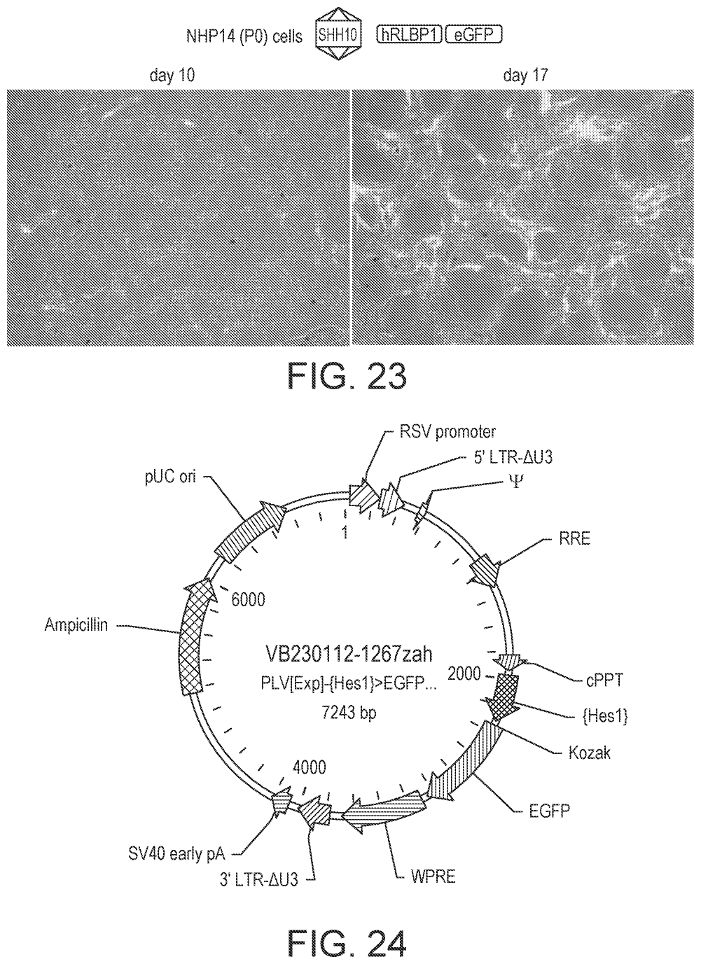

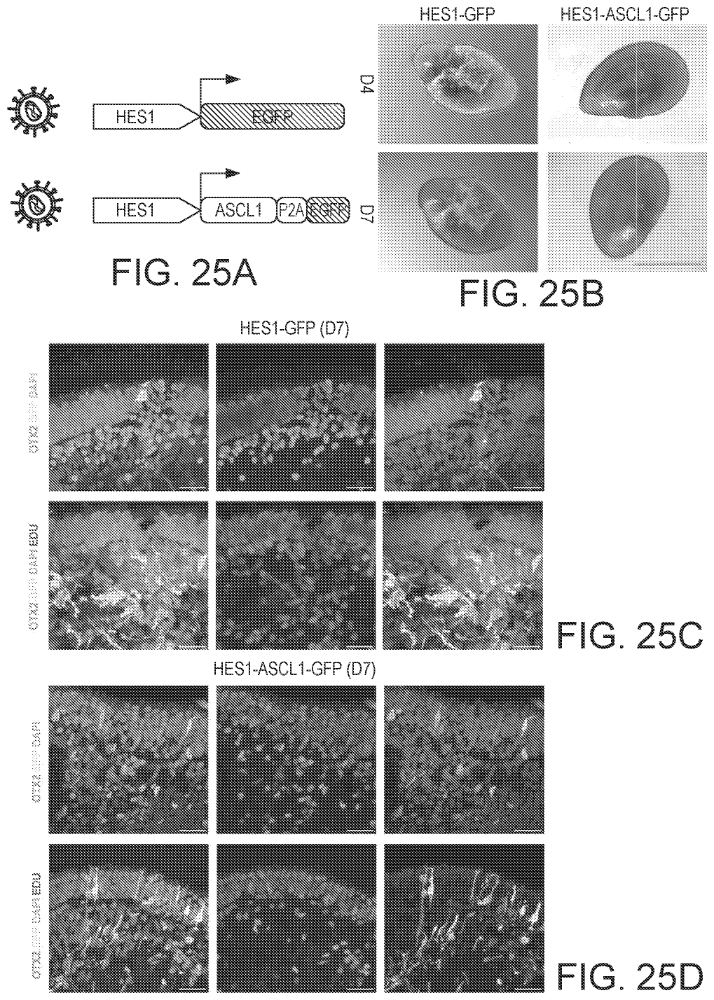

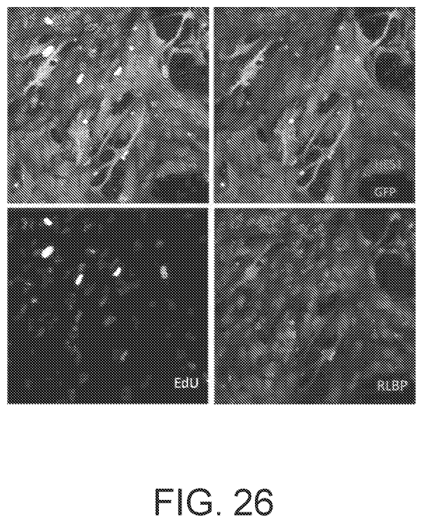

The characterization of ShH10 capsid and RLBP1 promoter in non-human primate (NHP) Muller glia is illustrated in FIG. 23. We have found that the human RLBP1 promoter works very well to drive expression in Muller glia (mouse, human or NHP), but, it is 2.8 kB and too cumbersome for 2-3 reprogramming TFs. Thus we began working with a much smaller promoter. HES1. HES1 promoter provides a good alternative to RLBP1 promoter. HES1 promoter is only 337 bp, it is expressed in adult Muller glia, and in retinal progenitors. In addition, HES1 is increased in expression after ASCL1 infection. As shown in FIG. 25, HES1 promoter drives very good expression specifically in Muller glia in human retinal organoids. GFP is expressed in HES1 and Sox9 positive cells seven days after the infection with the Hes1-GFP construct. The construct is specific at 7 days. FIG. 25C shows that GFP is not expressed in OTX2 positive cells (HES1-GF), however we do obtain GFP/OTX2 positive cells after the expression of ASCL1 (reprogrammed cells; FIG. 25D). HES1-promoter also directs GFP expression in MG in adult NHP dissociated cultures, as shown in FIG. 26.

This small and specific promoter offers the potential for a single AAV virus to drive reprogramming TFs in Muller glia. We made the viruses shown in FIG. 24, along with a more complicated “auto-regulatory loop” virus to allow us to track the reprogrammed cells over time. The HES1 promoter shows specificity. The related HESS offers another alternative.

Throughout this application various publications are referenced. The disclosures of these publications in their entireties are hereby incorporated by reference into this application in order to describe more fully the state of the art to which this invention pertains.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A nucleic acid molecule comprising a nucleic acid sequence encoding a developmental retinal ganglion cell (RGC) transcription factor, and
a promoter sequence in operable linkage with the nucleic acid sequence encoding the RGC transcription factor,
wherein the RGC transcription factor is selected from the group consisting of Onecut1, Pou4f2, Islet1, Irx2, Irx5, Neurod2, Ebf1, Tcf3, and combinations thereof; and
wherein the promoter sequence is HES1, RLBP1 or GLAST.

2. The nucleic acid molecule of claim 1, wherein the RGC transcription factor is selected from the group consisting of Onecut1, Pou4f2, and Islet1, and combinations thereof.

3. The nucleic acid molecule of claim 1, wherein the RGC transcription factor is Onecut1.

4. The nucleic acid molecule of claim 1, wherein the RGC transcription factor comprises Pou4f2 and/or Islet1.

5. The nucleic acid molecule of claim 1, wherein the RGC transcription factor is selected from the group consisting of Irx2, Irx5, Neurod2, Ebf1, Tcf3, and combinations thereof.

6. The nucleic acid molecule of claim 1, wherein the RGC transcription factor comprises Irx2 and/or Neurod2.

7. The nucleic acid molecule of claim 1, wherein the nucleic acid sequence further comprises a nucleic sequence that encodes a proneural basic helix-loop-helix (bHLH) transcription factor selected from Ascl1, Atoh1, and Atoh7.

8. (canceled)

9. The nucleic acid molecule of claim 8, wherein the promoter sequence further comprises a retinal ganglion cell (RGC)-specific promoter.

10. (canceled)

11. A composition comprising a nucleic acid molecule of claim 1.

12. The composition of claim 11, further comprising a histone deacetylase (HDAC) inhibitor.

13. The composition of claim 12, wherein the HDAC inhibitor is trichostatin A (TSA), (Pro)/romidepsin, (Pro)/belinostat, (Pro)/panobinostat, and/or (Pro)/vorinostat.

14. A method for stimulating regeneration of retinal ganglion cells in a subject, the method comprising: administering to a retina of the subject a composition of claim 11, thereby stimulating regeneration of retinal ganglion cells.

15. The method of claim 14, wherein the composition comprises a first nucleic acid molecule encoding a proneural bHLH transcription factor selected from the group consisting of Ascl1, Atoh1, or Atoh7, and a second nucleic acid molecule encoding a RGC transcription factor selected from the group consisting of Onecut1, Pou4f2, Islet1, Irx2, Irx5, Neurod2, Ebf1, Tcf3, and combinations thereof.

16. The method of claim 14, wherein the administering comprises a first administration of composition comprising a first nucleic acid molecule encoding a proneural bHLH transcription factor selected from the group consisting of Ascl1, Atoh1, or Atoh7, and a second administration at a subsequent time point of a composition comprising a second nucleic acid molecule encoding a RGC transcription factor selected from the group consisting of Onecut1, Pou4f2, Islet1, Islet1, Irx2, Irx5, Neurod2, Ebf1, Tcf3, and combinations thereof.

17. The method of claim 14, wherein the subject is treated for retinal disease, damage or degeneration in the retina.

18. The method of claim 14, wherein the subject is an adult.

19. (canceled)

20. (canceled)

21. The method of claim 20, wherein the nucleic acid molecule is an adeno-associated viral (AAV) vector or a lentiviral vector.

22. The method of claim 14, wherein the administering to the retina is intravitreal or subretinal injection.

23. The method of claim 15, wherein the proneural bHLH transcription factor and the RGC transcription factor are expressed as a fusion protein.

24. The method of claim 15, wherein the first and/or second nucleic acid molecule is an mRNA.

25. (canceled)
